(19) United States
(12) Patent Application Publication
(10) Pub. No.: US 2025/0327114 A1
(43) Pub. Date: Oct. 23, 2025

(54) BARCODE DETECTION USING ARGONAUTE PROTEINS

(71) Applicant: 10x Genomics, Inc., Pleasanton, CA (US)

(72) Inventors: Patrick J. MARKS, Paris (FR); Ruijie ZHANG, Milpitas, CA (US)

(21) Appl. No.: 19/186,302

(22) Filed: Apr. 22, 2025

Related U.S. Application Data
(60) Provisional application No. 63/637,841, filed on Apr. 23, 2024.

(51) Int. Cl. C12Q 1/6825; C12N 15/10; C12Q 1/6841; C12Q 1/6855

(52) U.S. Cl. CPC C12Q 1/6825; C12N 15/1093; C12Q 1/6841; C12Q 1/6855

(57) ABSTRACT

The present disclosure relates in some aspects to methods for analyzing target nucleic acids and their spatial locations in a biological sample using Argonaute proteins. In some aspects, a barcode probe library comprising a plurality of probes each comprising a plurality of barcode subunits that identifies a target analyte is detected in situ in the sample. Also provided are compositions and kits for use in accordance with the methods.

### CROSS-REFERENCE TO RELATED APPLICATIONS

This application claims benefit of and priority to U.S. Provisional Application No. 63/637,841, filed on Apr. 23, 2024, entitled “Barcode Detection Using Argonaute Proteins,” which is herein incorporated by reference in its entirety for all purposes.

### FIELD

The present disclosure relates in some aspects to methods and compositions for in situ analysis of nucleic acids in biological samples.

### BACKGROUND

Methods are available for detecting nucleic acids present in a biological sample. For instance, advances in single molecule fluorescent in situ hybridization (smFISH) have enabled nanoscale-resolution imaging of RNA in cells and tissues. However, barcode design and detection can be challenging for detecting a large panel of analytes. Improved methods for detecting barcode sequences in cell or tissue samples are needed. Provided herein are methods, compositions, and kits that address such and other needs.

Argonaute proteins are a large family of proteins derived from prokaryotic and eukaryotic organisms that use nucleic acid guides to target other nucleic acids. Some Argonaute family members use RNA guide nucleic acids, and some use DNA guide nucleic acids. In some aspects, the guide nucleic acid directs Argonaute binding to a target sequence complementary to the guide nucleic acid or a portion thereof (e.g., complementary to a seed sequence of the guide nucleic acid) with high sensitivity and specificity. Some Argonaute proteins lack nuclease activity. For such nuclease-deficient Argonaute proteins, the guide nucleic acid directs Argonaute binding to a specific nucleic acid sequence complementary to the guide nucleic acid sequence. In certain cases, Argonaute proteins are engineered to be nuclease-deficient.

### SUMMARY

The methods, systems, and kits described herein harnesses the sequence-specific binding activities of Argonaute proteins for improved methods of in situ detection. In some aspects, the guide nucleic acid-mediated sequence-specific binding properties of nuclease-deficient Argonaute are used to improve methods of detecting barcode sequences (e.g., in a rolling circle amplification product).

Provided herein is a method for detecting a target analyte in a biological sample comprising contacting the biological sample comprising a plurality of target analytes with a barcode probe library to provide a plurality of barcode probes, wherein each barcode probe of the barcode probe library comprises (i) a plurality of barcode subunits and (ii) a region that binds to a target analyte of the plurality of target analytes, wherein each barcode subunit is 10-30 nucleotides in length and the plurality of barcode subunits of the barcode probe library has a total of at least 50 different barcode subunits, wherein the plurality of barcode subunits on a barcode probe of the barcode probe library identifies the target analyte, and wherein each target analyte is assigned a signal code that identifies the target analyte; and detecting the plurality of barcode subunits in the probes bound to target analytes in a plurality of detection cycles using a plurality of barcode-binding probes to obtain the signal code, wherein each detection cycle comprises contacting the biological sample with at least a subset of the plurality of barcode-binding probes, wherein each barcode-binding probe of at least the subset of the plurality of barcode-binding probes is in a complex with a nuclease-deficient Argonaute protein and each barcode-binding probe comprises a barcode-binding domain that binds to a sequence of the barcode subunit of the plurality of barcode subunits of a barcode probe or a complement thereof; and detecting a signal associated with a bound barcode-binding probes to obtain a signal of the signal code; thereby determining the identity of the target analyte using the detected signal code. In some embodiments, each barcode subunit of the barcode probes of the barcode probe library is between 10-20 nucleotides in length.

In some embodiments, the barcode probe library has a total of at least 60, at least 100, or at least 160 different barcode subunits. In some embodiments, the barcode probe library has a total of at least 500 different barcode subunits. In some embodiments, the barcode probe library has a total of at least 1,000 different barcode subunits.

In some embodiments, the nuclease-deficient Argonaute protein is a eukaryotic Argonaute protein. In some embodiments, the plurality of barcode-binding probes further comprises additional barcode-binding probes that are not in a complex with Argonaute proteins. In some embodiments, the nuclease-deficient Argonaute protein is a DNA-guided Argonaute, and the barcode probes of the barcode probe library comprise DNA. In some embodiments, the nuclease-deficient Argonaute protein is a prokaryotic Argonaute protein. In some embodiments, the nuclease-deficient Argonaute protein is Ago1 or Ago4. In some embodiments, the nuclease-deficient Argonaute protein is a Drosophila Argonaute protein or a derivative or variant thereof. In some embodiments, the nuclease-deficient Argonaute protein is a nuclease-deficient Argonaute derived from Thermus thermophilus (dTtA go). In some embodiments, the nuclease-deficient Argonaute protein comprises one or more inactivating mutations in a PIWI and/or PAZ domain of the Argonaute protein.

In some embodiments, the barcode-binding probe and the nuclease-deficient Argonaute protein are bound in the complex before contacting the biological sample. In some embodiments, the barcode-binding probe and the nuclease-deficient Argonaute protein form a complex in the biological sample.

In some embodiments, the plurality of barcode subunits comprise artificial sequences with less than 70% homology to an endogenous human or mouse sequence. In some embodiments, the endogenous human or mouse sequence is a highly abundant sequence with a copy number of at least 1,000 or more copies per cell. In some embodiments, the endogenous human or mouse sequence is a DNA sequence. In some embodiments, the endogenous human or mouse sequence is an RNA sequence. In some embodiments, the endogenous human or mouse sequence comprises rRNA or tRNA. In some embodiments, the endogenous human or mouse sequence comprises a sequence of a centromere, a telomere, a SINE or a LINE.

In some embodiments, the nuclease-deficient Argonaute protein is labeled with a detectable moiety, optionally wherein the detectable moiety is a fluorescent dye. In some embodiments, the barcode-binding probes are labeled with a detectable moiety, In some embodiments, the detectable moiety is a fluorescent dye. In some embodiments, the barcode-binding probes are not directly labeled with a fluorescent dye. In some embodiments, the barcode-binding probes comprise a 3′ tail sequence, and wherein the method comprises contacting the biological sample with a detectably labeled probe that binds directly or indirectly to the 3′ tail sequence, and wherein the detecting comprises detecting the detectably labeled probe bound directly or indirectly to the barcode-binding probes.

In some embodiments, at least two different probes of the barcode probe library share a barcode subunit with the same sequence. In some embodiments, at least two barcode subunits of the plurality of barcode subunits within a barcode probe of the barcode probe library are overlapping. In some embodiments, the plurality of barcode subunits of each barcode probe of the barcode probe library are overlapping. In some embodiments, the plurality of barcode subunits of each barcode probe of the barcode probe library are overlapping by one or more nucleotides. In some embodiments, the plurality of barcode subunits of each barcode probe of the barcode probe library are overlapping by no more than 10 nucleotides. In some embodiments, at least two barcode subunits of the plurality of barcode subunits within a barcode probe of the barcode probe library are overlapping by no more than 10 nucleotides. In some embodiments, the plurality of barcode subunits of each barcode probe of the barcode probe library are overlapping. In some embodiments, at least two barcode subunits of the plurality of barcode subunits within a barcode probe of the barcode probe library are partially overlapping. In some embodiments, the plurality of barcode subunits of each barcode probe of the barcode probe library are partially overlapping such that at least one nucleotide is not overlapping between a first barcode subunit and a second barcode subunit. In some embodiments, the sequence overlapping between a first pair of barcode subunits and a second pair of barcode subunits comprises the same sequence.

In some embodiments, the detection comprises contacting the biological sample with a first subset of barcode-binding probes in a first detection cycle and subsequently contacting the biological sample with a second subset of barcode-binding probes in a second detection cycle, wherein the first subset of barcode-binding probes and second subset of barcode-binding probes share at least one barcode-binding probe with the same barcode-binding domain. In some embodiments, the detection comprises contacting the biological sample with a first subset of barcode-binding probes in a first detection cycle and subsequently contacting the biological sample with a second subset of barcode-binding probes in a second detection cycle, wherein the first subset of barcode-binding probes comprises at least one barcode-binding probe that does not have the same barcode-binding domain as a barcode-binding probe of the second subset of barcode-binding probes.

In some embodiments, the method comprises washing the biological sample between contacting the biological sample with different subsets of barcode-binding probes from the plurality of barcode-binding probes. In some embodiments, the washing is performed under less than stringent conditions.

In some embodiments, the method comprises generating a plurality of amplification products of the plurality of probes bound to the target analytes before detecting the plurality of barcode subunits. In some embodiments, the method comprises circularizing the plurality of probes bound to target analytes prior to generating the plurality of amplification products. In some embodiments, the 3′ end and the 5′ end of a probe of the plurality of probes are ligated to form a circularized probe. In some aspects, the plurality of barcode probes of the barcode probe library are a plurality of padlock probes. In some embodiments, the plurality of probes are ligated to form a circularized probe (e.g., circularized padlock probes).

In some embodiments, the plurality of amplification products is generated using a polymerase. In some embodiments, the polymerase is a Phi29 polymerase. In some embodiments, the plurality of amplification products are a plurality of rolling circle amplification products (RCPs). In some embodiments, the barcode-binding domain binds to a sequence of the barcode subunit in an RCP of the plurality of RCPs.

In some embodiments, the barcode-binding domain is between about 14 and 20 nucleotides in length. In some embodiments, the difference in length of the barcode-binding domains of the plurality of barcode-binding probes is no more than 4 nucleotides. In some embodiments, the barcode-binding domain of the plurality of different barcode-binding probes is the same number of nucleotides.

In some embodiments, detecting of the plurality of barcode subunits comprises imaging the biological sample.

In some embodiments, the plurality of target analytes targeted by the barcode probe library is at least 200 target analytes. In some embodiments, the plurality of target analytes targeted by the barcode probe library is at least 240 target analytes. In some embodiments, the plurality of target analytes targeted by the barcode probe library is at least 500 target analytes. In some embodiments, the plurality of target analytes targeted by the barcode probe library is at least 1,000 target analytes. In some embodiments, the plurality of target analytes targeted by the barcode probe library is at least 2,000 target analytes.

In some embodiments, the detecting is performed on a cell or tissue sample. In some embodiments, the plurality of target analytes comprise a plurality of cellular RNA analytes or a product thereof. In some embodiments, the plurality of target analytes are associated with a non-nucleic acid analyte. In some embodiments, the plurality of barcode probes of the barcode probe library binds to a plurality of oligonucleotide reporters, wherein each oligonucleotide reporter is in a labeling agent that binds to the target analyte. In some embodiments, the target analytes are mRNA. In some embodiments, the target analytes are cDNA.

In some embodiments, the biological sample is a tissue section. In some embodiments, the biological sample is a formalin-fixed, paraffin-embedded (FFPE) sample or a fresh frozen tissue sample. In some embodiments, the biological sample is a fresh frozen tissue sample. In some embodiments, the biological sample is fixed and/or permeabilized. In some embodiments, the biological sample is crosslinked and/or embedded in a matrix. In some embodiments, the matrix comprises a hydrogel. In some embodiments, the biological sample is cleared.

Provided herein is a kit comprising a barcode probe library to provide a plurality of probes bound to target analytes, wherein each barcode probe of the barcode probe library comprises (i) a plurality of barcode subunits and (ii) a region that binds to a target analyte; wherein each barcode subunit is 10-30 nucleotides in length and the plurality of barcode subunits of the barcode probe library has a total of at least 50 different barcode subunits; and a plurality of barcode-binding probes, wherein each barcode-binding probe is in a complex with a nuclease-deficient Argonaute protein and each barcode-binding probes comprises a barcode-binding domain that binds to a sequence of the barcode subunit of the plurality of barcode subunits of a barcode probe or a complement thereof.

In some embodiments, the barcode probe library has a total of at least 60, at least 100, or at least 160 different barcode subunits. In some embodiments, the barcode probe library has a total of at least 500 different barcode subunits. In some embodiments, the barcode probe library has a total of at least 1,000 different barcode subunits. In some embodiments, the Argonaute protein is a eukaryotic Argonaute protein. In some embodiments, the kit comprises a plurality of detectably labeled probes that binds directly or indirectly to a subset of the barcode-binding probe. In some embodiments, the plurality of barcode-binding probes further comprises additional barcode-binding probes that are not in a complex with Argonaute protein.

In some embodiments, the Argonaute protein is a DNA-guided Argonaute, and the barcode probes of the barcode probe library comprise DNA. In some embodiments, the Argonaute protein is a prokaryotic Argonaute protein. In some embodiments, the nuclease-deficient Argonaute protein is Ago1 or Ago4. In some embodiments, the nuclease-deficient Argonaute protein is a Drosophila Argonaute protein or a derivative or variant thereof. In some embodiments, the nuclease-deficient Argonaute protein is a nuclease-deficient Argonaute derived from Thermus thermophilus (dTtA go). In some embodiments, the nuclease-deficient Argonaute protein comprises one or more inactivating mutations in a PIWI and/or PAZ domain of the Argonaute protein.

In some embodiments, the barcode-binding domain is between about 10 and 20 nucleotides in length. In some embodiments, the difference in length of the barcode-binding domains of the plurality of barcode-binding probes is no more than 4 nucleotides. In some embodiments, the barcode-binding domain of the plurality of different barcode-binding probes is the same number of nucleotides.

In some aspects, provided herein is a system, comprising: a biological sample; a barcode probe library comprising a plurality of barcode probes bound to target analytes, wherein each barcode probe of the barcode probe library comprises (i) a plurality of barcode subunits and (ii) a region that binds to a target analyte; wherein each barcode subunit is 10-30 nucleotides and the plurality of barcode subunits of the barcode probe library has a total of at least 50 different barcode subunits; and a plurality of barcode-binding probes, wherein each barcode-binding probe is in a complex with a nuclease-deficient Argonaute protein and each barcode-binding probe comprises a barcode-binding domain that binds to a sequence of the barcode subunit of the plurality of barcode subunits of a barcode probe or a complement thereof. In some embodiments, the barcode probe library has a total of at least 60, at least 100, or at least 160 different barcode subunits. In some embodiments, the barcode probe library has a total of at least 500 different barcode subunits. In some embodiments, the barcode probe library has a total of at least 1,000 different barcode subunits.

In some embodiments, the Argonaute protein is a eukaryotic Argonaute protein. In some embodiments, the system further comprises a plurality of detectably labeled probes that binds directly or indirectly to a subset of the barcode-binding probe. In some embodiments, the plurality of barcode-binding probes further comprises additional barcode-binding probes that are not in a complex with the Argonaute protein. In some embodiments, the Argonaute protein is a DNA-guided Argonaute, and the barcode probes of the barcode probe library comprise DNA. In some embodiments, the Argonaute protein is a prokaryotic Argonaute protein. In some embodiments, the nuclease-deficient Argonaute protein is Ago1 or Ago4. In some embodiments, the nuclease-deficient Argonaute protein is a Drosophila Argonaute protein or a derivative or variant thereof. In some embodiments, the nuclease-deficient Argonaute protein is a nuclease-deficient Argonaute derived from Thermus thermophilus (dTtA go). In some embodiments, the nuclease-deficient Argonaute protein comprises one or more inactivating mutations in a PIWI and/or PAZ domain of the Argonaute protein. In some embodiments, the barcode-binding domain is between about 10 and 20 nucleotides in length. In some embodiments, the difference in length of the barcode-binding domains of the plurality of barcode-binding probes is no more than 4 nucleotides. In some embodiments, the barcode-binding domain of the plurality of different barcode-binding probes is the same number of nucleotides in length for each of the plurality of different barcode-binding probes.

### BRIEF DESCRIPTION OF THE DRAWINGS

The drawings illustrate certain features and advantages of this disclosure. These embodiments are not intended to limit the scope of the appended claims in any manner.

FIG. 1A shows an example method of detecting a plurality of barcode subunits at a location in the biological sample. A probe or probe set comprising barcode subunits targeting a target nucleic acid (e.g., RNA) is circularized and amplified. Sequences (e.g., barcode subunits) in the generated RCP are detected by performing at least two cycles of imaging to detect binding of barcode-binding probes each in a complex with an Argonaute protein to the barcode subunits.

FIG. 1B depicts various labeled Argonaute-barcode-binding probe complexes.

FIG. 2 shows a schematic illustration of an example of overlapping barcode subunits.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Available methods of in situ detection based on binding of probes to barcode sequences continue to face technical challenges. In some cases, it is desirable to provide a library of barcode probes, wherein each barcode probe of the barcode probe library comprises a plurality of barcode subunits, and to identify the different barcode probes by sequentially detecting the different barcode subunits. By sequentially detecting multiple different barcode subunits, a large number of different barcode probes can be decoded using only a small number of different barcode-binding probes. The challenges associated with such methods include the ability to design distinct barcode subunits such that each barcode subunit has high affinity for its correct barcode-binding probe, and sufficiently low affinity for other barcode-binding probes such that off-target binding is limited. This problem becomes more difficult as the number of distinct barcode subunits to be decoded in each cycle increases, and as the length of the barcode subunits decreases (which is desirable to reduce the length required for each barcode subunit, allowing for more barcode subunits to be included in a probe without increasing the cost for nucleic acid synthesis).

The present application harnesses the sequence-specific binding activities of Argonaute proteins for improved methods of in situ detection. In some aspects, the guide nucleic acid-mediated sequence-specific binding properties of nuclease-deficient Argonaute are used to improve methods of detecting barcode sequences (e.g., in a rolling circle amplification product). In some aspects, an Argonaute protein and barcode-binding probe complex is used to detect a barcode subunit. In some embodiments, the sequence-specific binding activity of the Argonaute protein improves specificity of barcode subunit binding for an Argonaute and barcode binding probe complex compared to a barcode binding probe alone.

Argonaute proteins are a large family of proteins that use nucleic acid guides to target other nucleic acids and either bind or cut at a defined location in a target sequence in the target nucleic acid. Argonaute family members are derived from prokaryotic and eukaryotic organisms. Some Argonaute family members use RNA guides as guide nucleic acids. Some Argonaute family members use DNA guides as guide nucleic acids. Some Argonaute family members bind RNA. Some Argonaute family members bind and cut RNA. Some Argonaute family members bind, but do not cut, RNA. Some Argonaute family members bind DNA. Some Argonaute family members bind and cut DNA. Some Argonaute family members bind, but do not cut, DNA. Argonaute proteins that cut a target nucleic acid are said to have slicer activity. Not all Argonaute proteins have slicer activity; for example, Argonaute proteins involved in miRNA-mediated post-transcriptional regulation are slicer-dead (i.e., the Argonaute-guide nucleic acid binds, but does not cut, at the target sequence). While Argonaute proteins are endogenously involved in gene regulation and defense from pathogenic sequences, Argonaute proteins have been demonstrated to be useful tools for molecular biology. In some embodiments, modified Argonaute proteins that lack slicer activity can be generated. In some aspects, complexes of slicer-dead (i.e., catalytically inert or nuclease-dead) Argonaute proteins with a nucleic acid guide are useful for improving hybridization events, such as compared to hybridization of free oligonucleotides. In some cases, complexes of Argonaute proteins with a nucleic acid guide hybridize to target sites faster than free oligonucleotides competing for the same target sites. In some cases, complexes of Argonaute proteins with a nucleic acid guide have a very low rate of off-target binding. This binding accuracy is due to the high sensitivity of the guide nucleic acid seed region (i.e., the seed region comprising nucleotides 2-8 at the 5′ end of the guide nucleic acid) to single-nucleotide mismatches. The guide nucleic acid requires full sequence complementarity to the target strand throughout the seed region. For some Argonaute proteins (e.g., such as non-cutting Argonaute proteins involved in regulation of miRNAs), sequence complementarity of a supplementary 3′ region with the nucleic acid target is also required for successful binding of the Argonaute-guide nucleic acid complex in addition to complementarity of the seed sequence.

In some embodiments, hybridization events using a probe in a complex with a nuclease-deficient Argonaute protein is highly specific due to the requirement for exact sequence complementarity within all or a part of the seed region of the nucleic acid probe (e.g., serving as a guide nucleic acid) in a complex with the Argonaute protein. In some embodiments, the seed region of the nucleic acid probe comprises 5′ nucleotides 2-8. In some embodiments, most or all of the seed region of a probe in a complex with a nuclease-deficient Argonaute protein must be complementary to the target sequence (e.g., a sequence of the barcode subunit) in order for target recognition and binding of the Argonaute-guide nucleic acid complex to the target sequence to occur. In some cases, an assay for detecting a large panel of analytes involves a plurality of barcode subunits that are designed to correspond to particular target analytes. As the number of target analytes to be detected in a sample increases, the number of unique barcodes that meet various criteria for hybridization and detection become more difficult to design. Thus, barcode design and detection can be challenging for detecting a large panel of analytes and using a probe in a complex with a nuclease-deficient Argonaute protein provides increased specificity and/or efficiency for the hybridization event for barcode detection.

### II. Detecting Barcode Sequences Using Argonaute Complexes With Barcode-Binding Probes

Argonaute-mediated hybridization of a barcode-binding probe to a barcode subunit may offer several advantages. For example, in some cases, Argonaute-mediated hybridization of a barcode-binding probe to a barcode subunit occurs more rapidly than probe hybridization in the absence of an Argonaute protein. In some embodiments, requirements for complementarity of the barcode-binding probe to the barcode subunit provides more stringent matching criteria than hybridization of free oligonucleotide probes (e.g., not in a complex with an Argonaute protein), allowing for precise detection and discrimination of barcode subunit sequences that may share some sequence similarity.

Argonaute proteins can be nuclease-active (i.e., have slicer activity) or nuclease-deficient (i.e., lack slicer activity). In some embodiments, provided herein is a method comprising contacting a biological sample with a nuclease-deficient Argonaute protein in a complex with a barcode-binding probe. In some embodiments, the barcode-binding probe serves as a guide nucleic acid for the Argonaute protein. In some embodiments, the nuclease-deficient Argonaute protein comprises a detectable moiety such as a fluorescent label. In some embodiments, the barcode-binding probe comprises a detectable moiety. In some embodiments, the method comprises detecting the bound Argonaute protein in a complex with a barcode-binding probe at a location in the biological sample, thereby detecting the complementary sequence of the barcode-binding probe at the location in the biological sample.

In some embodiments, the complementary sequence of the barcode-binding probe is a sequence of the barcode subunit of the plurality of barcode subunits of a probe or a complement thereof. In some embodiments, a sequence of the barcode subunit of the plurality of barcode subunits of a probe or a complement thereof is incorporated into a rolling circle amplification product using probes or probe sets that are circularized and amplified to generate the rolling circle amplification product. In certain embodiments, a complementary sequence of the barcode subunit is generated in the rolling circle amplification product (RCP) using a circularized probe as a template, and the rolling circle amplification product comprises multiple copies of the sequence of the barcode subunit. In some embodiments, the rolling circle amplification is performed according to any of the embodiments described in Section II.B.

In some embodiments, the method provided herein comprises contacting an RCP generated in a biological sample with a barcode-binding probe in a complex with a nuclease-deficient Argonaute protein. In some embodiments, the barcode-binding probe and the nuclease-deficient Argonaute protein form a complex prior to contacting the biological sample. In some embodiments, the barcode-binding probe and the nuclease-deficient Argonaute protein complex is guided to bind with the RCP. In some embodiments, the complex of barcode-binding probe and the nuclease-deficient Argonaute protein does not cut the RCP after the complex contacts the RCP.

### A. Barcode Subunits and Barcode-Binding Probes

Provided herein is a method for detecting a target analyte in a biological sample using barcode-binding probes in a complex with a nuclease-deficient Argonaute protein. In some embodiments, a biological sample comprises a plurality of target analytes and a barcode probe library comprising a plurality of barcode probes are used to bind to the target analytes. In some embodiments, each barcode probe of the barcode probe library comprises (i) a plurality of barcode subunits and (ii) a region that binds to a target analyte of the plurality of target analytes. In some aspects, the plurality of barcode subunits on a barcode probe of the barcode probe library identifies a target analyte, and wherein each target analyte is assigned a signal code that identifies the target analyte. As the number of analytes increases, the number of different barcode subunits needed to identify a target analyte increases. In some embodiments, each of the barcode subunits is at least 10 nucleotides and the plurality of barcode subunits of the barcode probe library has a total of at least 50 different barcode subunits. In some embodiments, the plurality of barcode subunits in the barcode probes bound to target analytes are detected in a plurality of detection cycles using a plurality of barcode-binding probes to obtain the signal code. For example, a detection cycle comprises: contacting the biological sample with at least a subset of barcode-binding probes from the plurality of barcode-binding probes, wherein each barcode-binding probe is in a complex with a nuclease-deficient Argonaute protein and each barcode-binding probes comprises a barcode-binding domain that binds to a sequence of the barcode subunit of the plurality of barcode subunits of a barcode probe or a complement thereof; and detecting a signal associated with a bound barcode-binding probes to obtain a signal of the signal code.

In some embodiments, hybridization events using a barcode-binding probe in a complex with a nuclease-deficient Argonaute protein is highly specific due to the requirement for exact sequence complementarity within all or a part of the seed region of the nucleic acid probe (e.g., barcode-binding probe serving as a guide nucleic acid) in a complex with the Argonaute protein. In some embodiments, a barcode-binding probe in a complex with a nuclease-deficient Argonaute protein must be complementary to the target sequence (e.g., a sequence of the barcode subunit) in order for target recognition and binding of the Argonaute-nucleic acid complex to the target sequence to occur. In some cases, an assay for detecting a large panel of analytes involves a plurality of barcode subunits that in combination are designed to identify a target analyte. In some embodiments, most or all of the seed region of a barcode-binding probe in a complex with a nuclease-deficient Argonaute protein is complementary to a sequence in a barcode subunit that is unique among the plurality of barcode subunits. In some embodiments, the plurality of barcode subunits comprise common overlapping sequences, and most or all of the seed region of a barcode-binding probe in a complex with a nuclease-deficient Argonaute protein is complementary to a sequence in a barcode subunit that is not part of the common overlapping sequences. In some embodiments, at least 1, 2, 3, 4, 5, or 6 nucleotides of the seed region is complementary to a sequence in a barcode subunit that is unique among the plurality of barcode subunits. In some embodiments, the seed region of the barcode-binding probe is nucleotides 2-8 of the barcode-binding probe, wherein the numbering is from the 5′ end of the barcode-binding probe. In some embodiments, the last 2-5 nucleotides at a 3′ end of a barcode subunit are a common sequence that is present in a different barcode subunit of the plurality of barcode subunits.

As the number of target analytes to be detected increases, the number of distinct barcode subunits that meet various criteria for hybridization and detection become more difficult to design. For example, it may be challenging to design a plurality of barcode subunits of a fixed length as the number of distinct barcode subunits in the barcode probe library increases. Thus, barcode subunit design and detection can be challenging for detecting a large panel of analytes and using a barcode-binding probe in a complex with a nuclease-deficient Argonaute protein provides increased specificity and/or affinity for the hybridization event for barcode detection. In some embodiments, using a barcode-binding probe in a complex with a nuclease-deficient Argonaute protein increases the binding stability and/or the binding duration of hybridization for barcode detection. In some embodiments, using a barcode-binding probe in a complex with a nuclease-deficient Argonaute protein provides increased efficiency (e.g., faster binding kinetics) for the hybridization needed for barcode detection. In some cases, it is desirable to design a barcode probe library wherein the plurality of barcode subunits of the probes have high affinity for the correct barcode-binding probe and low binding affinity for all other barcode-binding probes. In some aspects, it is desirable to design a barcode probe library wherein the melting temperatures (Tₘ) of the plurality of barcode subunit sequences for binding to incorrect barcode-binding probes is kept sufficiently low. In some embodiments, using a barcode-binding probe in a complex with a nuclease-deficient Argonaute protein has the effect of lowering the Tm of partially mismatched probes. In some aspects, using a barcode-binding probe in a complex with a nuclease-deficient Argonaute protein increases the difference in Tₘ between the correct interactions of barcode subunits of the probes with its respective barcode-binding probe and an incorrect interaction of a barcode subunit of the probes with a barcode-binding probe (e.g., a non-matching barcode-binding probe).

In some embodiments, barcode-binding probes exhibit some level of off-target binding (e.g., binding to a non-matching sequence of the barcode subunit that is less than 100% complementary). In some embodiments, each of the plurality of barcode-binding probes may exhibit at least about 1%, 2%, 3%, 4% or 5% off-target binding activity. In some embodiments, each of the plurality of barcode-binding probes may exhibit no more than about 5%, no more than about 4%, no more than about 3%, no more than about 2%, or no more than about 1%, off-target binding activity. In some embodiments, each of the plurality of barcode-binding probes may exhibit between about 1% to 5% off-target binding activity. In some cases, a barcode-binding probe in a complex with a nuclease-deficient Argonaute protein reduces the likelihood of off-target binding. In some aspects, off-target binding rate for a free barcode-binding probe (e.g., not in complex with an Argonaute protein) is higher than the off-target binding rate for a barcode-binding probe with the same sequence in a complex with a nuclease-deficient Argonaute protein.

In some embodiments, the barcode probe library has a total of at least 50, 60, 70, 80, 90, 100, 110, 120, 130, 140, 150, 160, 170, 180, 190, or 200 or more different barcode subunits. In some embodiments, the barcode probe library has a total of at least 200, 500, 600, 700, 800, 900, or 1,000 or more different barcode subunits. In some embodiments, the barcode probe library has a total of at least 60 or more different barcode subunits. In some embodiments, the probe library has a total of at least 100 or more different barcode subunits. In some embodiments, the probe library has a total of at least 140 or more different barcode subunits. In some embodiments, the probe library has a total of at least 160 or more different barcode subunits. In some embodiments, the probe library has a total of at least 250 or more different barcode subunits. In some embodiments, the probe library has a total of at least 500 or more different barcode subunits. In some embodiments, the probe library has a total of at least 750 or more different barcode subunits. In some embodiments, the probe library has a total of at least 1,000 or more different barcode subunits. In some embodiments, the probe library has a total of between about 50 and about 500, between about 50 and about 400, between about 50 and about 300, between about 50 and about 200, between about 50 and about 100 nucleotides, between about 100 and about 1,000, between about 100 and about 750, between about 100 and about 500, between about 100 and about 400, between about 100 and about 300, between about 100 and about 200, between about 150 and about 500, between about 150 and about 400, between about 150 and about 300, between about 150 and about 200, between about 250 and about 500, between about 500 and about 750, or between about 500 and about 1,000 different barcode subunits.

In some embodiments, the barcode-binding domain of the barcode-binding probe is at least about 5, at least about 8, at least about 10, at least about 12, at least about 15, at least about 20, or at least about 30 nucleotides in length. In some embodiments, the barcode-binding domain of the barcode-binding probe is at least 10 nucleotides in length. In some embodiments, the barcode-binding domain of the barcode-binding probe is between about 10 and about 30, about 15 and about 25, about 14 and about 20, about 16 and about 20, about 20 and about 30 nucleotides, or about 25 and about 35 nucleotides in length. In some embodiments, the barcode-binding domain of the barcode-binding probe is at least about 5, at least about 8, at least about 10, at least about 12, at least about 15, at least about 20, or at least about 30 nucleotides in length. In some embodiments, the barcode-binding domain of the barcode-binding probe is between about 10 and about 30, about 15 and about 25, about 14 and about 20, about 16 and about 20, about 20 and about 30 nucleotides, or about 25 and about 35 nucleotides in length. In some embodiments, the barcode-binding domain of the barcode-binding probe is 10 to 30, nucleotides in length 10 to 35 nucleotides in length, 20 to 35 nucleotides in length, 20 to 31 nucleotides in length, 20 to 25 nucleotides in length, 25-35 nucleotides in length, or 26 to 31 nucleotides in length. In some embodiments, the barcode-binding domain of the barcode-binding probe is 10 to 30 nucleotides in length. In some embodiments, the barcode-binding domain of the barcode-binding probe is 15 to 25 nucleotides in length. In some embodiments, the barcode-binding domain of the barcode-binding probe is 20 to 30 nucleotides in length. In some embodiments, the barcode-binding domain of the barcode-binding probe is 20 to 25 nucleotides in length. In some embodiments, the barcode-binding domain of the barcode-binding probe is 26 to 31 nucleotides in length. In some embodiments, the barcode-binding domain of the barcode-binding probe is fully complementary to the sequence of the barcode subunit. In some embodiments, the barcode-binding domain of the barcode-binding probe is partially complementary to the sequence of the barcode subunit. In some embodiments, the barcode-binding domain of the barcode-binding probe is at least about 60%, about 70%, about 80%, about 90%, about 95%, or about 100% complementary to the sequence of the barcode subunit. In some embodiments, the barcode-binding domain of two barcode-binding probes share some sequence similarity. In some embodiments, the barcode-binding domain of two barcode-binding probes share at least about 10%, about 20%, about 30%, about 40%, about 45%, or about 50% identity. In some embodiments, the barcode-binding domain of at least two barcode-binding probes share at least about 20% identity. In some embodiments, the barcode-binding domain of at least two barcode-binding probes share at least about 10%-20%, about 10%-30%, about 20%-30%, about 20%-40%, about 5%-25%, or about 5%-10% identity.

In some embodiments, each barcode subunit of a single probe or probe set is less than or about 15 nucleotides in length. In some embodiments, each barcode subunit of a single probe or probe set is less than or about 20 nucleotides in length. In some embodiments, each barcode subunit of a single probe or probe set is 10, 11, 12, 13, 14, 15, 16, 17, 18, 19, or 20 nucleotides in length.

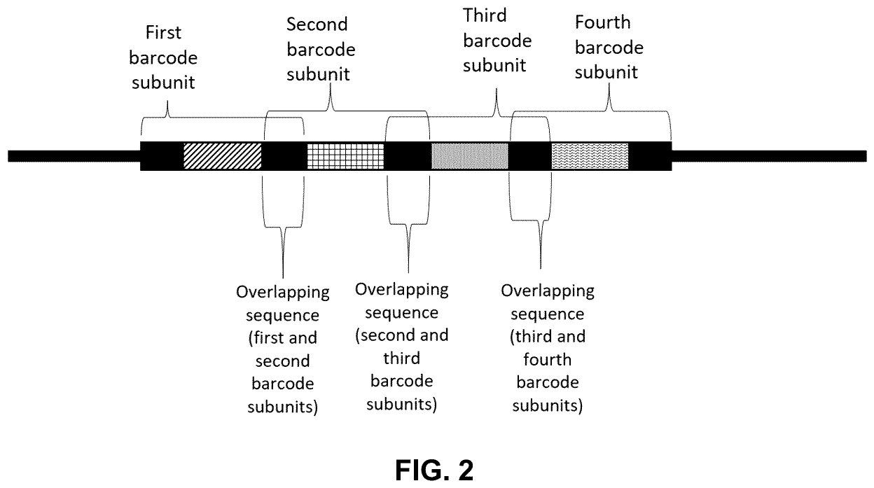

In some embodiments, a barcode subunit comprises a variable sequence. In some embodiments, a barcode subunit comprises a variable sequence comprising 5-12 nucleotides, optionally 7 nucleotides. In some embodiments, a barcode subunit comprises a constant or a repeating sequence unit. In some embodiments, the constant or repeating sequence comprises 4 nucleotides, wherein the constant or repeating sequence unit is present at regularly spaced intervals in the barcode probe. In some embodiments, the constant or repeating sequence unit comprises the nucleotide sequence CA CA. In some embodiments, a barcode probe comprises one or more variable sequences of 7 nucleotides each, wherein each variable sequence is flanked by a constant or repeating sequence unit. See, for example, FIG. 2. In some embodiments, a first barcode subunit comprises, from 5′ to 3′, a constant sequence unit, a first variable sequence, and a constant sequence unit, and a second barcode subunit comprises, from 5′ to 3′, the constant sequence unit at the 3′ end of the first barcode subunit, a second variable sequence, and a constant unit sequence.

In some embodiments, the barcode-binding domain of a plurality of different barcode-binding probes (e.g., with different sequences) is about the same length. In some embodiments, the barcode-binding domain of a plurality of different barcode-binding probes (e.g., with different sequences) is the same number of nucleotides. In some embodiments, the different barcode subunits (e.g., with different sequences) used in a barcode probe library is about the same length. In some embodiments, the length of different barcode subunits (e.g., with different sequences) used in a barcode probe library is longer or shorter by no more than 1, 2, 3, 4, or 5 nucleotides. In some embodiments, the length of the barcode-binding domain of a plurality of different barcode-binding probes (e.g., with different sequences) is longer or shorter by no more than 1, 2, 3, 4, or 5 nucleotides. In some embodiments, the difference in length of the barcode-binding domains of a plurality of different barcode-binding probes (e.g., with different sequences) is no more than 4 nucleotides. In some embodiments, the difference in length of the barcode-binding domains of a plurality of different barcode-binding probes (e.g., with different sequences) is no more than 3 nucleotides. In some embodiments, the difference in length of the barcode-binding domains of a plurality of different barcode-binding probes (e.g., with different sequences) is no more than 2 nucleotides. In some embodiments, the difference in length of the barcode-binding domains of a plurality of different barcode-binding probes (e.g., with different sequences) is no more than 1 nucleotide.

In some aspects, the potential interaction(s) between two or more nucleic acid strands are analyzed. For example, modeling software is used, in some cases, to predict potential interactions between two or more nucleic acid strands. In some instances, a plurality of nucleic acid strands comprises (i) a plurality of probes each comprising a plurality of barcode subunits or complementary sequences thereof and (ii) a plurality of barcode-binding probes. In some instances, a plurality of nucleic acid strands comprises probes comprising a plurality of barcode subunits or complementary sequences thereof, a plurality of barcode-binding probes and a plurality of endogenous sequences (e.g., DNA, RNA). In some embodiments, the endogenous sequences are a highly abundant biological sequences found in a biological sample, for example rRNA, tRNA, centromere, telomere, SINE and/or LINE. In some embodiments, the endogenous sequences each have a copy number of 1,000 or more copies per cell. In some embodiments, the endogenous sequence is a DNA with a copy number of 1,000 or more copies per cell. In some embodiments, the endogenous sequence is a RNA with a copy number of 1,000 or more copies per cell. In some embodiments, the endogenous sequence has a copy number of 1,000 or more copies per cell in the biological sample. In some embodiments, the copy number is an expected copy number of the sequence in the cells of the biological sample. In some embodiments, the plurality of barcode subunits of a barcode probe library comprises artificial sequences that have less than 90%, 95%, 80%, 85%, 80%, 75%, 70%, 65%, 55%, or 50% homology to an endogenous human or mouse sequence. In some embodiments, the plurality of barcode subunits of a barcode probe library comprises artificial sequences that have less than 70% homology to an endogenous human or mouse sequence. In some embodiments, the plurality of barcode subunits of a barcode probe library comprise artificial sequences that have less than 90%, 95%, 80%, 85%, 80%, 75%, 70%, 65%, 55%, or 50% homology to any “highly abundant” endogenous biological sequence. In some embodiments, the plurality of barcode subunits of a barcode probe library comprise artificial sequences that have less than 70% homology to any “highly abundant” endogenous biological sequence. In some embodiments, the plurality of barcode subunits of a barcode probe library comprise artificial sequences that have less than 70% homology to any endogenous human or mouse DNA and/or RNA sequence. In some embodiments, the plurality of barcode subunits of a barcode probe library comprise artificial sequences that have less than 70% homology to any endogenous human or mouse rRNA, tRNA, centromere, telomere, SINE or LINE. In some embodiments, the plurality of barcode subunits of a barcode probe library comprise artificial sequences that have less than 70% homology to any endogenous human or mouse DNA and/or RNA sequence with a copy number of 1,000 or more copies per cell. In some embodiments, the plurality of barcode subunits of a barcode probe library comprise artificial sequences that have less than 70% homology to any endogenous human or mouse DNA and any endogenous human or mouse RNA sequence with a copy number of 1,000 or more copies per cell. Suitable tools for determining whether a designed artificial sequence has less than 70% homology to any endogenous human or mouse DNA and any endogenous human or mouse RNA sequence with a copy number of 1,000 or more copies per cell include, but are not limited to, NCBI nucleotide BLAST (blastn) suite.

In some aspects, the potential interaction(s) between sequences of probes or complementary sequences thereof in a barcode probe library and a plurality of barcode-binding probes are analyzed. In some aspects, the potential interaction(s) between barcode subunit sequences in an amplification product of a probe and a plurality of barcode-binding probes are analyzed. In some aspects, the potential interaction(s) between endogenous sequence, sequences of barcode probes in a barcode probe library and a plurality of barcode-binding probes are analyzed. In some aspects, the potential interaction(s) between endogenous sequences, barcode subunit sequences in an amplification product of a barcode probe and a plurality of barcode-binding probes are analyzed.

For example, NUPACK is a software suite for analyzing and designing various nucleic acid structures, devices, and systems. In some cases, NU PACK algorithms treat complex and test tube ensembles with a plurality of interacting strand species and provide tools to capture concentration effects essential to analyzing and designing the intermolecular interactions. In some cases, NUPACK is used to analyze interactions for scalable large complexes. See Fornace et al, NUPACK: analysis and design of nucleic acid structures, devices, and systems; ChemRxiv, 10.26434/chemrxiv-2022-xv98I, 2022. In some examples, the analysis of potential interaction(s) involving sequences of probes in a barcode probe library (or complementary sequences thereof) and a plurality of barcode-binding probes uses an algorithm which models interactions between “strands” (e.g., a single-stranded DNA/RNA molecule with a fixed sequence). In some examples, the analysis of potential interaction(s) involving sequences of barcode probes in a barcode probe library and a plurality of barcode-binding probes uses an algorithm which models interactions of “tubes” (e.g., a mixture of different oligos at known concentrations provided as a set of [nucleic acid strand, concentration] pairs). In some examples, the analysis of potential interaction(s) involving sequences of barcode probes in a barcode probe library and a plurality of barcode-binding probes uses an algorithm which models formations of “complexes” (e.g., a set of strands bound together via some base-pairing interactions). In some examples, the analysis of potential interaction(s) involving sequences of probes in a barcode probe library (or complementary sequences thereof) and a plurality of barcode-binding probes uses an algorithm which models for a given complex, all the possible conformations and structures that can be formed. In some examples, the analysis of potential interaction(s) involving sequences of probes in a barcode probe library (or complementary sequences thereof) and a plurality of barcode-binding probes uses an algorithm which models for a given complex, all the possible conformations and structures that can be formed, including partial and not fully paired (e.g., between not completely complementary sequences) structures.

In some embodiments, the analysis of potential interaction(s) involving sequences of barcode probes in a barcode probe library (or complementary sequences thereof) and a plurality of barcode-binding probes takes into consideration of any mismatch, insertions or deletions in the sequences being analyzed. In some cases, the analysis of potential interaction(s) involving sequences of barcode probes in a barcode probe library and a plurality of barcode-binding probes takes into consideration of the position of any mismatch, insertion or deletion in the sequences being analyzed. In some aspects, the analysis of potential interaction(s) involving sequences of barcode probes in a barcode probe library and a plurality of barcode-binding probes considers melting temperatures (Tₘ) of the sequences. For example, in some cases, the analysis of potential interaction(s) involving sequences of barcode probes in a barcode probe library (or complementary sequences thereof) and a plurality of barcode-binding probes takes into consideration that a mismatch at the end of a sequence causes less Tₘ reduction than a mismatch in the middle of the sequence.

In some embodiments, the analysis of potential interaction(s) involving sequences of probes in a barcode probe library (or complementary sequences thereof) and a plurality of barcode-binding probes provides a prediction of how strongly two candidate sequences will interact. In some cases, the prediction of potential interaction(s) involving sequences of barcode probes in a barcode probe library and a plurality of barcode-binding probes takes into consideration properties of the sequences analyzed, including sequence composition of the sequences, the length of the sequences, and/or the position of the nucleotides within the oligonucleotide.

In some embodiments, the analysis of potential interaction(s) involving sequences described herein is used to screen sequences for use as the plurality of barcode subunits in a barcode probe library. In some embodiments, the analysis of potential interaction(s) involving sequences is used to evaluate the interaction of a tube of nucleic acid strands A and B. In some cases, the analysis provides that three potential complexes form: A-only, B-only, and A+B bound together. In some embodiments, the analysis of potential interaction(s) involving sequences is used to evaluate the interaction of a tube of nucleic acid strands and a specified temperature. In some embodiments, the analysis of potential interaction(s) provides the concentrations of all the complexes that will form.

In some examples, an analysis of potential interaction(s) comprises the input of a tube with 1 strand representing an amplification product (e.g., RCP) of a barcode probe generated in a biological sample, and a plurality of barcode-binding probes. In some cases, the analysis provides the potential complexes that form from the tube (e.g., with the RCP and barcode-binding probes) and it is desirable if a matching barcode-binding probe forms a complex with the sequence of the barcode subunit or complement thereof at a high fraction of the RCP concentration and that complexes with non-matching barcode-binding probes are formed at low rates. In some embodiments, the selected barcode subunit sequences and barcode-binding probe sequences to be used in a complex with Argonaute proteins to improve binding have a higher tolerance for non-matching interaction(s) compared to sequences that are used as free oligonucleotides where binding occurs without the aid of the Argonaute protein.

In some examples, potential interaction(s) between a plurality of barcode-binding probes and a plurality of detectably labeled probes (e.g., that binds to the 3′ tail sequence of the barcode-binding probe) are analyzed. In some examples, potential interaction(s) between an amplification product (e.g., RCP) of a probe generated in a biological sample, a plurality of barcode-binding probes and a plurality of detectably labeled probes (e.g., that binds to the 3′ tail sequence of the barcode-binding probe) are analyzed.

In some embodiments, the barcode probe library and barcode-binding probes described herein is designed with barcode subunit sequences suitable for use under selected conditions (e.g., temperature, salt concentration, buffer compositions, and other reaction conditions). For example, such sequences are designed and selected using software for analyzing various nucleic acid structures (e.g., NUPACK). In some embodiments, the barcode probe library and barcode-binding probes described herein comprise barcode subunit sequences that exhibit low cross-reactivity. In some embodiments, the barcode probe library and barcode-binding probes described herein comprise barcode subunit sequences that exhibit similar melting temperatures. In some embodiments, the barcode probe library comprise a plurality of barcode subunit sequences with melting temperatures that deviate by no more than 1, 2, 3, 4, 5, 6, 7, 8, 9, or 10 degrees Celsius.

In some aspects, the present application provides designs for barcode-binding probes capable of forming complexes with the Argonaute protein to bind at least a portion of a sequence of the barcode subunit of the plurality of barcode subunits of a probe or a complement thereof in an amplification product of the probe. In some embodiments, the barcode-binding probes are used to achieve highly sensitive sequence binding, resulting in improved sensitivity (number of detected RCPs), signal intensity, increased positional stability in the biological sample, improved accuracy of localization, improved signal to noise, and homogeneity (e.g., narrower size and intensity distributions) compared to detection performed without Argonaute proteins.

In some embodiments, the barcode-binding probes comprise RNA. In some embodiments, the barcode-binding probes comprise DNA. In some embodiments, the barcode-binding probes comprise both DNA and RNA. In some embodiments, the barcode-binding probes are single-stranded. In some cases, the barcode-binding probes are single-stranded DNA (ssDNA) oligonucleotides. In some embodiments, the barcode-binding probes comprise one or more synthetic nucleotides and/or one or more synthetic nucleosides. In some embodiments, the one or more synthetic nucleosides comprise bromodeoxyuridine (BrdU).

In some embodiments, the barcode-binding probe is an RNA molecule, and the Argonaute protein is an RNA-guided Argonaute. In some embodiments, the barcode-binding probe is a DNA molecule, and the Argonaute protein is a DNA-guided Argonaute. In some embodiments, the subset of the plurality of the barcode-binding probes are distinguishable by an associated detectable label (e.g., a fluorescent label).

### B. Barcode Probe Library

Provided herein is a barcode probe library to provide a plurality of barcode probes for targeting a plurality of target analytes, wherein each barcode probe of the barcode probe library comprises a plurality of barcode subunits and a region that binds to a target analyte. In some embodiments, a hybridization product comprising the pairing of substantially complementary or complementary nucleic acid sequences within two different molecules is analyzed (e.g., between a probe and a target analyte). In some embodiments, pairing is achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another. In some embodiments, a hybridization product comprising a sequence of the barcode subunit or complementary nucleic acid sequences with a barcode-binding probe is analyzed. In some embodiments, the sequence of the barcode subunit is in a barcode probe of a barcode probe library or an amplification product generated using the probe as template.

In some embodiments, the sequence of the barcode subunit is in a probe bound to an endogenous target analyte. In some embodiments the sequence of the barcode subunit is in a probe bound to reporter oligonucleotide of a labeling agent. In some embodiments, the probes or probe sets is a circularizable probe or probe set. In some embodiments, the barcode probe library comprises a plurality of padlock probes. In some embodiments, the probe is based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, or RNA-templated ligation probes. The specific probe or probe set design can vary.

In some embodiments, provided are nucleic acids (e.g., barcode probes, barcode-binding probes) for binding to other nucleic acids. In some instances, the nucleic acids bind via hybridization, typically by Watson-Crick base pairing, such as DNA, RNA, LNA, PNA, etc., depending on the application. In some embodiments, nucleic acids (e.g., barcode probes, barcode-binding probes) are able to bind to at least a portion of another nucleic acid. In some embodiments, the nucleic acids (e.g., barcode probes, barcode-binding probes) bind to a specific target nucleic acid analyte. In some aspects, binding refers to the coupling between two or more nucleic acids, e.g., oligonucleotides and/or polynucleotides. In some embodiments, the binding is indirect binding. In some embodiments, the binding is direct (e.g., binding comprising direct hybridization of nucleic acid sequences). The nature of the binding may vary. In some instances, a first nucleic acid sequence directly binds to a second nucleic acid sequence via hybridization of complementary sequences. In some instances, a first nucleic acid sequence indirectly binds to a second nucleic acid sequence via one or more intermediate nucleic acids. For example, an intermediate nucleic acid comprises a first region that binds to the first nucleic acid sequence and has a second region for binding to the second nucleic acid sequence, thereby forming a complex comprising the first and second nucleic acid sequences.

### (a) Ligation

In some embodiments, a ligation product generated by the circularization of a circularizable probe or probe set (e.g., barcode probe or probe set comprising a plurality of barcode subunits) upon hybridization to a target sequence in the target analyte is detected. In some embodiments, a method comprises circularizing the plurality of probes bound to target analytes prior to generating a plurality of amplification products. In some aspects, the 3′ end and the 5′ end of a probe are bound to a target analyte and the ends are ligated to form a circularized barcode probe. In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 2019/0055594, which is hereby incorporated by reference in its entirety. In some embodiments, provided herein is a multiplexed proximity ligation assay. See, e.g., U.S. Pat. Pub. 2014/0194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 2016/0108458, which is hereby incorporated by reference in its entirety. In some embodiments, a circular probe is indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety. In some embodiments, any suitable probe or probe set is designed to comprise at least two barcode subunits.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NA D-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo) nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (b) Primer Extension and Amplification

In some embodiments, a primer extension product of a probe or probe set bound to the target analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) is analyzed.

A primer is generally a single-stranded nucleic acid sequence having a 3′ end that, in some embodiments, is used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis).

In some embodiments, the primer oligonucleotide for amplification of the circular or circularized probe (e.g., the circular or circularized barcode probe) comprises a single-stranded nucleic acid sequence having a 3′ end that is used as a substrate for a nucleic acid polymerase in a nucleic acid extension reaction. The primer oligonucleotide can comprise both RNA nucleotides and DNA nucleotides (e.g., in a random or designed pattern). The primer oligonucleotide can also comprise other natural or synthetic nucleotides described herein that can have additional functionality. Primers can vary in length. For example, primers is about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. In some embodiments, enzymatic extension is performed by an enzyme including, but not limited to, a polymerase and/or a reverse transcriptase.

In some embodiments, an amplification product of one or more polynucleotides, for instance, a circular barcode probe or circularizable barcode probe or probe set, is generated and detected. In some embodiments, the amplifying is achieved by performing rolling circle amplification (RCA). In some embodiments, an endogenous nucleic acid or fragment thereof hybridized to the circular barcode probe or circularized barcode probe is used to prime amplification. In some embodiments, a primer that hybridizes to the circular barcode probe or circularized barcode probe is added and used as such for amplification. In some embodiments, the RCA comprises a linear RCA, a branched RCA, a dendritic RCA, or any combination thereof.

In some instances, upon addition of a DNA polymerase in the presence of appropriate dNTP precursors and other cofactors, the amplification primer is elongated by replication of multiple copies of the template. In some embodiments, the amplification step utilizes isothermal amplification or non-isothermal amplification. In some embodiments, after the formation of the hybridization complex and any subsequent circularization (such as ligation of, e.g., a barcode probe or probe set), the circular probe is rolling-circle amplified to generate a RCA product (e.g., amplicon) containing multiple copies of the sequence of the circular probe.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

In some embodiments, the polymerase comprises a modified recombinant Phi29-type polymerase. In some embodiments, the polymerase comprises a modified recombinant Phi29, B103, GA-1, PZA, Phi15, BS32, M2Y, Nf, G1, Cp-1, PRD1, PZE, SF5, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Examples of polymerases are described in U.S. Pat. Nos. 8,257,954; 8,133,672; 8,343,746; 8,658,365; 8,921,086; and 9,279,155, all of which are herein incorporated by reference. In some embodiments, the polymerase is not directly or indirectly immobilized to a substrate, such as a bead or planar substrate (e.g., glass slide), prior to contacting a sample, although the sample may be immobilized on a substrate.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, amplification of a circular probe or circularizable probe (e.g., circular or circularizable barcode probe comprising a plurality of barcode subunits) or probe set is primed by the target RNA. The target RNA can optionally be immobilized in the biological sample. In some embodiments, the target RNA is cleaved by an enzyme (e.g., RNase H). In some embodiments, the target RNA is cleaved at a position downstream of the target sequences bound to the circular probe or circularizable probe or probe set. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. The cleaved target RNA itself can then be used to prime RCA of the circular probe generated from a circularizable probe or probe set (e.g., target-primed RCA). In some cases, a plurality of nucleic acid oligonucleotides are used to perform target-primed RCA for a plurality of different target RNA s.

In any of the embodiments herein, the biological sample is contacted with the RNase H (and optionally with the nucleic acid oligonucleotide) before or during formation of the circularized probe or probe set. In some embodiments, the biological sample is contacted with the oligonucleotide and with the RNase H simultaneously or sequentially (in either order) before contacting the sample with the probe or probe set. In any of the embodiments herein, the biological sample is contacted with the RNase H (and optionally with the nucleic acid oligonucleotide) after formation of the circularized probe or probe set. In any of the embodiments herein, the RNase H comprises an RNase H1 and/or an RNAse H2. In some embodiments, RNase inactivating agents or inhibitors are added to the sample after cleaving the target RNA.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:I095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides are added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) are anchored to a polymer matrix. For example, the polymer matrix can be a hydrogel. In some embodiments, one or more of the polynucleotide probe(s) is modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2017/079406, US 2016/0024555, US 2018/0251833, and US 2017/0219465, each of which are herein incorporated by reference in its entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

In some embodiments, the amplification products are immobilized within the matrix generally at the location of the nucleic acid being amplified, thereby creating a localized colony of amplicons. The amplification products may be immobilized within the matrix by steric factors. In some embodiments, the amplification products are immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a sequence thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. In some cases, the RCA template determines the signal which is detected, and is thus indicative of the target analyte.

In some embodiments, a target analyte described herein is bound to a probe (e.g., a circular or circularizable barcode probe or probe set) comprising a plurality of barcode subunits, e.g., at least two, three, four, five, six, seven, eight, nine, ten, or more barcodes. In some embodiments, a plurality of barcode subunits together function as a single barcode. For example, a barcode comprises two or more barcode subunit sequences that are separated by one or more non-barcode sequences. In some embodiments, two or more barcode subunit sequences are not separated by any non-barcode sequences. In some embodiments, the plurality barcode subunits of each probe of the barcode probe library are overlapping by one or more nucleotides. In some embodiments, the plurality of barcode subunits of each probe of the barcode probe library are overlapping by no more than 10 nucleotides. In some embodiments, the plurality of barcode subunits of each probe of the barcode probe library are overlapping by no more than 5 nucleotides. In some embodiments, the plurality of barcode subunits of a subset of probes of the barcode probe library are overlapping. In some embodiments, the plurality of barcode subunits of each probe of the barcode probe library are overlapping. In some embodiments, the plurality of barcode subunits of each probe of the barcode probe library are partially overlapping such that at least one nucleotide is not overlapping between a first barcode subunit and a second barcode subunit.

In some embodiments, the plurality barcode subunits within a probe of the barcode probe library are overlapping by one or more nucleotides. In some embodiments, the plurality of barcode subunits within a probe of the barcode probe library are overlapping by no more than 10 nucleotides. In some embodiments, the plurality of barcode subunits within a probe of the barcode probe library are overlapping by no more than 5 nucleotides. In some embodiments, the plurality of barcode subunits of a subset of probes of the barcode probe library are overlapping. In some embodiments, the plurality of barcode subunits within a probe of the barcode probe library are overlapping. In some embodiments, the plurality of barcode subunits within a probe of the barcode probe library are partially overlapping such that at least one nucleotide is not overlapping between a first barcode subunit and a second barcode subunit. In some embodiments, the two or more barcode subunit sequences are overlapping. In some embodiments, the two or more barcode subunit sequences are overlapping by at least 1, at least 2, at least 3, at least 4, at least 5, at least 6, at least 7, at least 8 nucleotides. In some embodiments, the two or more barcode subunit sequences are overlapping by no more than 10 nucleotides, no more than 8 nucleotides, no more than 6 nucleotides, or no more than 4 nucleotides. In some embodiments, the two or more barcode subunit sequences are overlapping by between 2-10 nucleotides, between 3-8 nucleotides, or between 4-6 nucleotides. In some embodiments, the barcode probe comprises at least three barcode subunit sequences, and the barcode subunit sequence overlaps with the adjacent barcode subunit sequence by at least 1, at least 2, at least 3, at least 4, at least 5, at least 6, at least 7, at least 8 nucleotides. In some embodiments, the sequence overlapping between a first pair of barcode subunits and a second pair of barcode subunits comprises the same sequence. In some embodiments, the barcode subunits are used for detection and identification of the probe or probe set. In any of the preceding embodiments, the methods provided herein can include analyzing the barcode subunits by sequential hybridization and detection with a plurality of barcode-binding probes.

In some embodiments, the combination of barcode subunits in a particular probe or probe set is used for identification of the probe. In some embodiments, a barcode subunit with the same sequence is used in two different probes, wherein the two different probes each comprise another barcode subunit with a different sequence. Thus, while individual barcode subunits can be shared among at least two different probes of the barcode probe library, the combination of barcode subunits in a probe is unique to the target analyte bound by the probe. In some embodiments, at least two different probes of the barcode probe library share the same barcode subunit. In some embodiments, a plurality of barcode subunits on each probe of the barcode probe library are overlapping. In some embodiments, the sequence overlapping between a first pair of barcode subunits in a single probe or probe set and a second pair of barcode subunits in the same single probe or probe set comprises the same sequence. In some embodiments, the probes of the barcode probe library comprise at least three barcode subunits per probe or probe set that share an overlapping sequence. An example of multiple barcode subunits that share an overlapping sequence is shown in FIG. 2. In some embodiments, as shown in FIG. 2, a first barcode subunit overlaps with a second barcode subunit. In some embodiments, a second barcode subunit overlaps with a third barcode subunit. In some embodiments, a third barcode subunit overlaps with a fourth barcode subunit. In some embodiments, the probe or probe set comprises additional barcode subunits and overlapping sequences (e.g., 5 or more overlapping barcode subunits). In some embodiments, two overlapping barcode subunits that are each 15 nucleotides in length have 4 nucleotides of overlap. In some embodiments, barcode subunits within a probe or probe set comprises a common domain at the 5′ and 3′ end of the barcode subunit. In some embodiments, the common domain at the 5′ and 3′ end of the barcode subunit is 2 nucleotides, 3 nucleotides, 4 nucleotides, 5 nucleotides, 6 nucleotides, 7 nucleotides, 8 nucleotides, 9 nucleotides or 10 nucleotides. In some embodiments, the common domain at the 5′ and 3′ end of the barcode subunit is no more than 5 nucleotides.

### C. Nuclease-Deficient Argonaute Proteins

In some embodiments, the method presented herein comprises detecting barcode subunit sequences with an Argonaute-barcode-binding probe complex comprising a barcode-binding probe and an Argonaute protein that does not have cutting activity (i.e., an Argonaute that is slicer-dead). Any suitable Argonaute protein for binding a nucleic acid in a nucleic acid duplex without cutting can be used. Generally, Argonaute proteins contain 6 main domains (N-terminal, L1 (Linker 1), PAZ (Piwi-Argonaute-Zwille), L2 (Linker 2), MID (Middle) and PIWI (P-element induced wimpy testis) responsible for binding of a guide nucleic acid and recognition of a guide target sequence. In some embodiments, the Argonaute protein is an RNA-guided Argonaute, and the guide nucleic acid is an RNA molecule. In some embodiments, the Argonaute protein is a DNA-guided Argonaute, and the guide nucleic acid is a DNA molecule.

In some embodiments, the Argonaute protein is a naturally-occurring protein (e.g., naturally occurs in prokaryotic or eukaryotic cells). In some embodiments, the Argonaute protein is not a naturally-occurring protein (e.g., a variant or mutant protein). In some embodiments, the Argonaute protein is a recombinant protein. In some embodiments, the Argonaute protein is genetically engineered (such as an Argonaute protein described in WO 2019/222036, which is hereby incorporated by reference in its entirety). In some embodiments, the Argonaute protein is a slicer-dead Argonaute protein, meaning that it lacks cutting activity or is nuclease-dead. In some embodiments, the Argonaute protein has been modified (e.g., genetically engineered or mutated) to lack cutting activity. In some embodiments, lacking cutting activity means that the Argonaute protein is not capable of cutting a target nucleic acid. In some embodiments, lacking cutting activity means that the Argonaute protein does not cut the target nucleic acid. In some embodiments, an Argonaute protein that naturally lacks cutting activity or that has been modified to lack cutting activity is a slicer-dead Argonaute.

In some embodiments, the Argonaute protein is a eukaryotic Argonaute protein. Generally, eukaryotic Argonaute proteins can mediate binding of a target RNA with a guide nucleic acid of RNA. In some embodiments, an Argonaute protein is of plant, algal, fungal (e.g., yeast), or animal (e.g., human, rodent, fruit fly, cnidarian, echinoderm, nematode, fish, amphibian, reptile, bird, etc.) origin. In some embodiments, the Argonaute protein is a eukaryotic Argonaute protein that has been modified to lack cutting activity.

In some embodiments, the Argonaute protein is a slicer-dead Ago1, Ago2, Ago3, Ago4, PIWI 1, PIWIL 2, PIWI 3, or PIWI 4 (such as the Argonaute proteins described in WO 2007/048629, which is hereby incorporated by reference in its entirety). In some embodiments, the Argonaute protein is Ago2. In some embodiments, the Ago2 is Drosophila Ago2. In some embodiments, the Argonaute protein is a recombinant Drosophila Argonaute protein. In some embodiments, the Argonaute protein is expressed in a mammalian cell line. In some embodiments, the Argonaute protein is a Drosophila Argonaute protein expressed in a mammalian cell line. In some embodiments, a Drosophila Argonaute protein is expressed using a method such that a loading complex specific to Drosophila species is not provided to obtain guide-free proteins. In some embodiments, the Argonaute protein is a purified recombinant Drosophila Argonaute protein. In some embodiments, the Argonaute protein is expressed in an insect cell line, such as a Schneider 2 (S2) cell line. In some embodiments, the Argonaute protein is a Drosophila Argonaute protein expressed in an insect cell line, such as a S2 cell line. In some embodiments, the Drosophila Argonaute protein is loaded with the barcode-binding probe prior to contacting the biological sample.

In some embodiments, the slicer-dead Argonaute protein is a eukaryotic Argonaute protein from a mammalian organism. In some embodiments, the mammalian Argonaute protein is selected from mammalian AGO1, AGO2, AGO3, and AGO4. In some embodiments, the mammalian Argonaute protein is a human Argonaute protein. In some embodiments, the human Argonaute protein is a human AGO1 or AGO4 protein which naturally lacks slicer activity (See Faehnle et al. The making of a slicer: activation of a human Argonaute-1. Cell Reports 2015 June 27, 3(6): 1901-1909, which is hereby incorporated by reference in its entirety). In some embodiments, the human Argonaute protein is a human AGO2 protein that has been modified to lack slicer activity (See McGeary et al., The Biochemical Basis of microRNA Targeting Efficacy. Science 2019 Dec 20; 366(6472): eaav1741., which is hereby incorporated by reference in its entirety). In some embodiments, the human Argonaute protein is a human AGO3 protein that has been modified to lack slicer activity.

In some embodiments, the Argonaute protein is used as described herein for binding a sequence of the barcode subunit of the plurality of barcode subunits of a probe or a complement thereof at a temperature at which slicer activity of the Argonaute protein is not active. In some examples, an Argonaute protein derived from Thermus thermophilus (dTtA go) is used to bind a target sequence at about 30° C. (See Shin et al, “Quantification of purified endogenous miRNAs with high sensitivity and specificity.” Nature Commun 11:6033 (2020), which is herein incorporated by reference in its entirety).

In some embodiments, the slicer-dead Argonaute protein is a prokaryotic Argonaute protein or a variant thereof. Generally, prokaryotic Argonaute proteins can mediate binding of a target RNA with a guide oligonucleotide. In some cases, the prokaryotic Argonaute protein uses RNA as the barcode-binding probe is in a complex with a nuclease-deficient Argonaute protein. In some cases, the prokaryotic Argonaute protein uses DNA. In some embodiments, the slicer-dead Argonaute protein is a prokaryotic Argonaute protein that has been modified to lack cutting activity.

In some embodiments, the slicer-dead Argonaute protein is a modified Nitratireductor (optionally Nitratirereductor sp. XY-223), Enhydrobacter (optionally Enhydrobacter aerosaccus), Mesorhizobium (optionally Mesorhizobium sp. CNPSo 3140), Hyphomonas (optionally Hyphomonas sp. T16B2), Pseudooceanicola (optionally Pseudooceanicola lipolyticus), Tateyamaria (optionally Tateyamaria omphalii), Bradyrhizobium (optionally Bradyrhizobium sp. ORS 3257), Dehalococcoides (optionally Dehalococcoides mccartyi), Chroococcidiopsis (optionally Chroococcidiopsis cubana), Runella (optionally Runella slithyformis), Roseivirga (optionally Rosevirga seohaensis), Spirosoma (optionally Spirosoma endophyticum), Pedobacter (optionally Pedobacter yonginense, Pedobacter insulae, or Pedobacter nyackensis), Planctomycetes bacterium (optionally Planctomycetes bacterium TBK1r or Planctomycetes bacterium V6), Dyadobacter (optionally Dyadobacter sp. QTA69), Mucilaginibacter (optionally Mucilaginibacter gotjawali, Mucilaginibacter polytichastri or Mucilaginibacter paludis), Hydrobacter (optionally Hydrobacter penzbergensis), Chitinophaga (optionally Chitinophaga costaii), Cytophagaceae bacterium (optionally Cytophagaceae bacterium SJ W1-29), Emticicia (optionally Emticicia oligotrophica), Runella (optionally Runella sp. YX9), or Spirosoma (optionally Spirosoma pollinicola) Argonaute protein (See Li et al., “A programmable pAgo nuclease with RNA target preference from the psychrotolerant bacterium Mucilaginibacter paludis” Nucleic Acids Res. 2022 May 20;50(9):5226-5238; Lisitskaya et al., “Programmable RNA targeting by bacterial Argonaute nucleases with unconventional guide binding and cleavage specificity.” Nat Commun. 2022 Aug 8;13(1):4624; Sun et al., “An Argonaute from Thermus parvatiensis exhibits endonuclease activity mediated by 5′ chemically modified DNA guides.” Acta Biochim Biophys Sin (Shanghai). 2022 May 25;54(5):686-695; U.S. Pat. No. 10,253,311;U.S. Pat. Pub. US2016/0289734;U.S. Pat. Pub. US2022/0186254; U.S. Pat. Pub US2022/0389425; and WO 2022/222920 each of which herein incorporated by reference in their entireties) that has been modified to lack cutting activity. In some embodiments, the slicer-dead Argonaute protein is from Thermus thermophilus. In some embodiments, the slicer-dead Argonaute protein is from Marinitoga piezophile (See Lapinaite et al, “Programmable RNA recognition by a CRISPR-associated Argonaute.” PNAS 2018 Mar 27;115(13):3368-3373, which is herein incorporated by reference in its entirety). In some embodiments, the slicer-dead Argonaute protein is from Rhobacter sphaeroidis (See Miyoshi et al, “Structural basis for the recognition of guide RNA and target DNA heteroduplex by Argonaute.” Nature Comm 2016; 7: 11846, which is herein incorporated by reference in its entirety). In some embodiments, the slicer-dead Argonaute protein is from Thermomyces thermophilus (such as an Argonaute protein described in patent application no. WO2023/138082, the content of which is herein incorporated by reference in its entirety). In some embodiments, the slicer-dead Argonaute protein is from Vanderwaltozyma polyspora (also known as Kluyveromyces polysporus) (such as an Argonaute protein described in WO 2018/112336, the content of which is herein incorporated by reference in its entirety). In some embodiments, the slicer-dead Argonaute protein is a modified Argonaute protein from Clostridium perfringens (CpAgo) or an Argonaute protein from Intestinibacter bartlettii (IbAgo) that lacks cutting activity (See Cao et al, Argonaute proteins from human gastrointestinal bacteria catalyze DNA-guided cleavage of single-and double-stranded DNA at 37 C. Cell Discovery 2019 5(38), which is hereby incorporated by reference in its entirety). In some embodiments, the slicer-dead Argonaute protein is a modified Argonaute protein from Clostridium butyricum (CbAgo) that lacks cutting activity (See Hegge et al. DNA-guided DNA cleavage at moderate temperatures by Clostridium butyricum Argonaute. BioRXIV 2019, and Kuzmenko et al. Programmable DNA cleavage by Ago nucleases from mesophilic bacteria Clostridium butyricum and Limnothrix rosea. BioRXIV 2019, both of which are hereby incorporated by reference in their entirety).

In some embodiments, the slicer-dead Argonaute protein is a variant of a DNA-binding Argonaute protein. In some embodiments, the Argonaute is a DNA-guided Pyrococcus furiosus (PfAgo) that binds single- and/or double-stranded DNA (See Swarts et al, “Argonaute of the archaeon Pyrocuccus furiosus is a DNA-guided nuclease that targets cognate DNA.” Nucleic Acids Research Volume 43, Issue 10, 26 May 2015, Pages 5120-5129, which is herein incorporated by reference in its entirety). In some embodiments, the Argonaute protein has been modified to lack cutting activity of an RNA substrate via selection and/or directed evolution.

In some embodiments, the slicer-dead Argonaute protein comprises one or more amino acid substitutions compared to any of the species of Argonaute protein described herein. In certain embodiments, the one or more amino acid substitutions are conservative substitutions. In some aspects, conservative amino acid substitutions can frequently be made in a protein without altering either the conformation or the function of the protein. Proteins in some cases comprise 1, 2, 3, 4, 5, 6, 7, 8, 9, 10, 11, 12, 13, 14, 15 conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g., U.S. Pat. No. 8,562,989; pages 13-15 of “Biochemistry” 2^{nd} ED. Lubert Stryer ed (Stanford University); Henikoff et al., PNAS, vol. 89, pp. 10915-10919 (1992); Lei et al., J. Biol. Chem, vol. 270, no. 20, pp.11882-11886 (1995).

An amino acid substitution may include replacement of one amino acid in a polypeptide with another amino acid. Amino acid substitutions may be introduced to generate a modified Argonaute protein as described herein.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some contexts, conservative substitutions can involve the exchange of a member of one of these classes for another member of the same class. In some contexts, non-conservative amino acid substitutions can involve exchanging a member of one of these classes for another class. In some contexts, particular substitutions can be considered “conservative” or “non-conservative” depending on the stringency and context and environment of the particular residue in primary, secondary and/or tertiary structure of the protein.

In some embodiments, the Argonaute is nuclease-deficient (i.e., lacks slicer activity). In some embodiments, the Argonaute is not capable of cutting the RCP. In some embodiments, the Argonaute protein does not cut the RCP. In some embodiments, the Argonaute protein is an Argonaute protein that lacks slicer activity. In some embodiments, the Argonaute protein is an Argonaute protein that has been modified (i.e., selectively mutated) to lack slicer activity. In some embodiments, the modified, nuclease-deficient Argonaute protein comprises one or more inactivating mutations in a PIWI and/or PAZ domain of the Argonaute protein.

In some embodiments, the slicer-dead Argonaute protein is an RNA-guided Argonaute, and the barcode-binding probe is an RNA molecule. In some embodiments, the slicer-dead Argonaute protein is a eukaryotic Argonaute protein. In some embodiments, the slicer-dead Argonaute protein is a DNA-guided Argonaute, and the barcode-binding probe is a DNA molecule. In some embodiments, the slicer-dead Argonaute protein is a prokaryotic Argonaute protein. In some embodiments, the barcode-binding probe comprises a 5′-phosphate or a 5′-OH. In some embodiments, the nuclease-deficient Argonaute protein is Ago1, Ago3, or Ago4. In some embodiments, the nuclease-deficient Argonaute protein is a Drosophila Argonaute protein or a derivative or variant thereof. In some embodiments, the nuclease-deficient Argonaute protein is a nuclease-deficient Argonaute derived from Thermus thermophilus (dTtA go).

In some embodiments, the nuclease-deficient Argonaute is a nuclease-deficient Argonaute derived from Marinitoga piezophile (MpAgo). In some embodiments, the nuclease-deficient Argonaute is a MpAgo that has been additionally modified to lack slicer activity. In some embodiments, the MpAgo protein forms a complex with a 5′-hydroxylated barcode-binding probe to form a MpAgo-barcode-binding probe complex. In some embodiments, the barcode-binding probe in the MpAgo complex comprises a 5′-BrdU (e.g., as described in Lapinaite et al., “Programmable RNA recognition using a CRISPR-associated Argonaute”, PNAS 2018) for increased binding stability of MpAgo-guide nucleic acid complex. In some embodiments, a seed region of the MpAgo-barcode-binding probe complex comprises a noncanonical seed region comprising nucleotides 5-15 of the barcode-binding probe. In some embodiments, the noncanonical 5-15 nucleotide seed region of the MpAgo-barcode-binding probe has full complementarity to the barcode subunit.

### D. In Situ Detection of Argonaute-Barcode-Binding Probe Complexes

In some embodiments, the method disclosed herein comprises contacting an RCP generated in a biological sample with an Argonaute-barcode-binding probe complex comprising a nuclease-deficient Argonaute protein (i.e., a slicer-dead Argonaute protein that lacks cutting activity) and a barcode-binding probe as described herein. In some embodiments, the contacting comprises contacting the biological sample with a plurality of nuclease-deficient Argonaute-barcode-binding probe complexes wherein the Argonaute-barcode-binding probe complexes comprise distinct barcode-binding domains that bind to a sequence of the corresponding barcode subunit of the plurality of barcode subunits of a probe or a complement thereof. In some embodiments, the Argonaute-barcode-binding probe complexes are distinct. In some embodiments, each different Argonaute-barcode-binding probe complex comprises a different detectable label. In some embodiments, the Argonaute-barcode-binding probe complex binds to the RCP at a sequence of the barcode subunit or complement thereof, enabling detection of the target analyte bound by the barcode probe (e.g., circular or circularizable barcode probe) in the biological sample. In some embodiments, at least one detecting cycle comprises contacting the biological sample with a plurality of barcode-binding probes that comprises a barcode-binding probe that is not in a complex with an Argonaute protein.

In some aspects, the provided methods involve analyzing, e.g., detecting or determining, one or more barcode subunits present in the probes or probe sets or products thereof (e.g., rolling circle amplification products thereof). In some embodiments, the detecting is performed at one or more locations in the biological sample. In some embodiments, the locations are the locations of RNA transcripts in the biological sample. In some embodiments, the locations are the locations at which the probes or probe sets hybridize to the RNA transcripts in the biological sample, and are optionally ligated and amplified by rolling circle amplification.

In some embodiments, the detecting comprises a plurality of repeated cycles of hybridization and removal of barcode-binding probes (e.g., in a complex with an Argonaute protein) to the probe or probe set hybridized to the target analyte, or to a rolling circle amplification product generated from the probe or probe set hybridized to the target analyte. In some embodiments, one or more barcode subunits present in the probes or probe sets or products thereof or any suitable amplification products generated therefrom (e.g., a branched DNA amplification product, a HCR reaction, etc.) are detected.

In some instances, the disclosed methods may comprise the use of a hybridization chain reaction (HCR) approach to amplify signals. In a hybridization chain reaction, two fluorescently-labeled metastable hairpin oligonucleotides self-assemble into long fluorescent polymers starting from an initiator sequence present on each probe molecule (Xia, et al. (2019), ibid.). The degree of amplification achieved through HCR can be tuned by changing the hybridization or polymerization times, and can be adjusted to achieve highly amplified signals (which may, however, increase the size of the fluorescent spots generated and/or lead to variable degrees of amplification for different copies of the same target molecule).

In some embodiments, provided herein are methods and compositions for analyzing analytes in a sample using concatemer primers and labeling agents. In various embodiments, a primer with domain on its 3′ end binds to a catalytic hairpin, and is extended with a new domain by a strand displacing polymerase. For example, a primer with domain 1 on its 3 ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer.

In various embodiments, a plurality of concatemer primers is contacted with a sample. In various embodiments, an assembly include a plurality of concatemer primers, a plurality of labeled probes, and a sample including nucleic acids. In various embodiments, each of the plurality of concatemer primers each includes domain 1, 2, 3, etc. In various embodiments, each the plurality of labeled probes each include domain 1′, 2′, 3′, etc., with each corresponding domain 1′, 2′, 3′ being complementary to domain 1, 2, 3, etc., respectively. In various embodiments, the assembly includes the plurality of concatemer primers, which are capable of hybridizing to target nucleic acid sequences in the sample. Described herein is a method using the aforementioned assembly, including contacting the sample including target nucleic acids with the plurality of concatemer primers, then contacting the sample and plurality of concatemer primers with the plurality of labeled probes, thereby labeling the target nucleic acid sequences with a plurality of labeled probes. See e.g., Kishi et al., SABER amplifies FISH: enhanced multiplexed imaging of RNA and DNA in cells and tissues, Nat. Methods. (2019) vol. 16, pp. 533-544; Saka et al., Immuno-SABER enables highly multiplexed and amplified protein imaging in tissues. Nat. Biotechnol. (2019) vol. 37, pp. 1080-1090, and U.S. Pat. Pub. No. 2021/0147902, each of which is fully incorporated by reference herein.

In some embodiments, detecting a plurality of barcode subunits in the barcode probes comprises detecting a signal directly or indirectly associated with a barcode-binding probe that is complementary to a barcode subunit of the plurality of barcode subunit. In some embodiments, the barcode-binding probe comprises a detectable moiety, optionally a fluorophore. In some embodiments, the nuclease-deficient Argonaute protein comprises a detectable moiety, optionally a fluorophore.

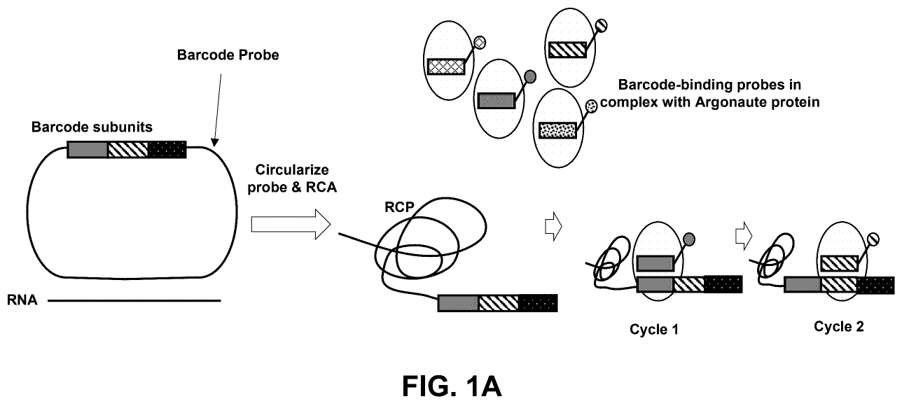

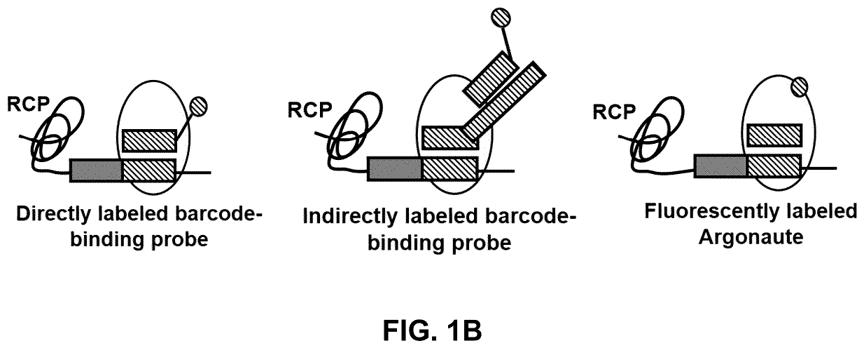

An example of a method of detecting bound barcode-binding probes in a complex with an Argonaute protein bound to an RCP at a location in the biological sample is shown in FIG. 1A. In some embodiments, a biological sample is contacted with a barcode probe library comprising a plurality of probes for binding to a plurality of target analytes (e.g., RNA as shown in FIG. 1A). In some examples, each barcode probe of the barcode probe library comprises (i) a plurality of barcode subunits and (ii) a region that binds to a target analyte of the plurality of target analytes, wherein each barcode subunit is 10-30 nucleotides in length and the plurality of barcode subunits of the barcode probe library has a total of at least 50 different barcode subunits. In some aspects, the plurality of barcode subunits on a barcode probe of the barcode probe library identifies the target analyte. In some cases, the barcode probes of the barcode probe library are circular probes. In some cases, the barcode probes of the barcode probe library are circularizable probes. In some cases, the barcode probe library comprises a plurality of barcode probe sets, each barcode probe set targeting a target analyte. In some cases, the barcode probe library comprises a plurality of circularizable barcode probe sets wherein each barcode probe set once bound to a target analyte is ligated (e.g., at both ends to the other molecule of the probe set) and forms a circularized barcode probe set. In some cases, the barcode probe library comprises a plurality of circularizable barcode probes. In some cases, once bound to the target analyte, the circularizable barcode probes are ligated to form circularized barcode probes. In some cases, the circular barcode probes, the barcode probe sets, or the ligated circularizable barcode probes are used as template for rolling circle amplification.

In some embodiments, a plurality of barcode-binding probes in a complex with a nuclease-deficient Argonaute protein as shown in FIG. 1A are used to detect the plurality of barcode subunits in the probes bound to the target analytes, or complements thereof in the rolling circle amplification product generated using the circularized probe or probe sets. For simplicity, detecting a “barcode subunit” herein refers to detecting either the barcode subunit itself or to detecting a complement of the barcode subunit, such as in an amplification product generated from a probe or probe set comprising the barcode subunit. For example, a first nuclease-deficient Argonaute protein in a complex with a first barcode-binding probe in detection cycle 1 binds to a complementary barcode subunit sequence in the generated RCP. A first signal associated with the bound first nuclease-deficient Argonaute protein in a complex with a first barcode-binding probe is detected and then removed from the RCP. In some embodiments, the barcode-binding probe of the first Argonaute-barcode-binding probe complex is labeled and detected. In some embodiments, a second nuclease-deficient Argonaute protein in a complex with a second barcode-binding probe in detection cycle 2 binds to a second complementary barcode subunit sequence in the RCP. A second signal associated with the bound second nuclease-deficient Argonaute protein in a complex with the second barcode-binding probe is detected and then removed from the RCP. In some embodiments, the biological sample comprising RCPs is imaged to detect the first fluorophore of the first bound Argonaute-barcode-binding probe complex at a location in the biological sample and the second fluorophore of the second bound Argonaute-barcode-binding probe complex at the same location in the biological sample, in sequential cycles. In some embodiments, detection of the first fluorophore and second fluorophore provides a first and second signal of the signal code and the identity of the target analyte is determined using at least the detected first and second signals.

In some embodiments, a nuclease-deficient Argonaute protein of an Argonaute-barcode-binding probe complex comprises a detectable moiety, for example, as shown in FIG. 1B (right panel). In some embodiments, the barcode-binding probe of the Argonaute-barcode-binding probe complex comprises a detectable moiety for example, as shown in FIG. 1B (left panel). In some embodiments, the barcode-binding probe of the Argonaute-barcode-binding probe complex is bound to a detectably labeled probe that comprises a detectable moiety for example, as shown in FIG. 1B (middle panel). In some embodiments, the detectably labeled probe is a detectably labeled probe that binds directly or indirectly with the 3′ tail sequence of the barcode-binding probe. In some embodiments, the detectable moiety comprises a detectable fluorescent label. In some embodiments, a plurality of detectable fluorescent labels are used to label the Argonaute-barcode-binding probe complex.

In some embodiments, the barcode-binding probe in a complex with a nuclease-deficient Argonaute protein is detected in situ with a detectably labeled probe comprising a sequence complementary to a portion of the barcode-binding probe. In some embodiments, the method comprises detecting the barcode-binding probe in a complex with a nuclease-deficient Argonaute protein by detecting a portion of the barcode-binding probe. In some embodiments, the barcode-binding probe in a complex with a nuclease-deficient Argonaute protein is a directly labeled barcode-binding probe. In some embodiments, the barcode-binding probe comprises a detectable moiety (e.g., a fluorescent label) as shown in the left panel of FIG. 1B. In some embodiments, the barcode-binding probe in a complex with a nuclease-deficient Argonaute protein is an indirectly labeled barcode-binding probe. In some embodiments, the barcode-binding probe in a complex with a nuclease-deficient Argonaute protein is detected in situ by binding a detectably labeled probe to an overhang of the barcode-binding probe, as shown in the middle panel of FIG. 1B.

In some embodiments, the barcode-binding probe in a complex with the nuclease-deficient Argonaute protein comprises an optional 3′ tail sequence in the overhang, wherein the method comprises contacting the RCP in the biological sample with a detectably labeled probe that binds directly or indirectly to the 3′ tail sequence of the bound barcode-binding probe, and wherein detecting the barcode-binding probe in a complex with the nuclease-deficient Argonaute protein bound to the RCP in the biological sample comprises detecting the detectably labeled probe bound directly or indirectly to the 3′ tail sequence of the barcode-binding probe. In some embodiments, the detectably labeled probe is a detectably labeled probe of a plurality of detectably labeled probes, wherein the method comprises contacting sequential cycles of binding barcode-binding probes to the plurality of barcode subunits of a probe or a complement thereof.

In some embodiments, a detectably labeled probe hybridizes to a portion of an Argonaute-barcode-binding probe complex disclosed herein.

In some embodiments, the overhang comprises a sequence associated with the barcode-binding domain of the barcode-binding probe. In some embodiments, the slicer-dead Argonaute protein comprises a detectable moiety (e.g., a fluorescent label) as shown in the right panel of FIG. 1B. In some embodiments, the method comprises detecting the detectable moiety of the slicer-dead Argonaute protein. In some embodiments, the slicer-dead Argonaute protein is contacted with the biological sample in a pre-formed complex with the barcode-binding probe, and the barcode-binding probe or detectable label attached to the slicer-dead Argonaute protein corresponds to a sequence of the barcode-binding probe. In some embodiments, the detectable label or detectably labeled probe is selected such that it identifies a sequence of barcode-binding probe that determines binding of the barcode-binding probe/Argonaute complex to a complementary sequence of the barcode subunit. In some aspects, as shown in FIG. 1B, Argonaute-barcode-binding probe complexes are (i) detectably labeled with a fluorescent moiety that corresponds to the specific barcode subunit on the barcode-binding probe, (ii) indirectly labeled using fluorescently labeled probes that bind to an overhang of the barcode-binding probe, or (iii) the Argonaute protein is detectably labeled with a fluorescent moiety that corresponds to the specific barcode subunit on the barcode-binding probe.

In some embodiments, a plurality of detectable fluorescent labels are used to label a plurality of barcode-binding probe that is in a complex with a nuclease-deficient Argonaute protein.

In some embodiments, a barcode-binding probe in a complex with the nuclease-deficient Argonaute protein is labeled with a detectable moiety such that it can be directly detected in situ in a biological sample. In some embodiments, the Argonaute protein is labeled with the detectable moiety. In some embodiments, the barcode-binding probe in a complex with the nuclease-deficient Argonaute protein is labeled with the detectable moiety. In some embodiments, the detectable moiety is a fluorescent dye. In some embodiments, the fluorescently labeled barcode-binding probe is detected at a location in the biological sample.

In some aspects, the provided methods involve analyzing, e.g., detecting or determining, one or more barcode subunit sequences. In some embodiments, the detection or determination comprises binding one or more Argonaute-barcode-binding probe complexes to nucleic acid molecules such as RCPs. In some cases, the analysis is performed on one or more images captured, and may comprise processing the image(s) and/or quantifying signals observed. In some embodiments, the analysis comprises detecting a sequence (e.g., a barcode subunit sequence) present in the sample. In some embodiments, the analysis comprises quantification of puncta (e.g., if amplification products are detected). In some embodiments, the obtained information is compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further comprises displaying the information from the analysis or detection step. In some embodiments, software may be used to automate the processing, analysis, and/or display of data.

In some embodiments, provided herein are methods involving the use of one or more probes for analyzing one or more target nucleic acid(s) in a cell or a biological sample, such as a tissue sample. In some embodiments, the barcode-binding probes can include a plurality of barcode-binding probes for combinatorially decoding the barcode subunits in the probes bound to target analytes or in the RCPs generated using probes bound to target analytes. Using sequential probe hybridization, the provided embodiments can be employed for in situ detection of barcode subunit sequences in probes bound to target analytes in a cell, e.g., in cells of a biological sample or a sample derived from a biological sample, such as a tissue section on a solid support, such as on a transparent slide.

In some aspects, provided herein are in situ assays using microscopy as a readout, e.g., hybridization, or other detection or determination methods involving an optical readout. In some aspects, detection or determination of a barcode subunit sequence is performed in situ in a cell in an intact tissue. In some aspects, detection or determination of a barcode subunit sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product or other amplification product). In some embodiments, the detecting comprises contacting the RCPs with a detectably labeled Argonaute-barcode-binding probe complex comprising a barcode-binding probe comprising a barcode-binding domain that binds to a sequence of the barcode subunit of the plurality of barcode subunits of a probe or a complement thereof, and then detecting the detectably labeled Argonaute-barcode-binding probe complex at the location in the biological sample. In some embodiments, a provided method is quantitative and preserves the spatial information within a tissue sample without physically isolating cells or using homogenates. In some embodiments, the present disclosure provides methods for high-throughput profiling of target nucleic acids in situ in a large number of cells, tissues, organs or organisms.

In some embodiments, the provided methods comprise imaging the amplification product (e.g., RCA product) of a probe or probe set (e.g., as described in Section II.B) and the bound Argonaute-barcode-binding probe complex, for instance, in sequential probe hybridization and detection cycles.

A method disclosed herein comprises contacting a biological sample with an plurality of Argonaute-barcode-binding probe complexes that binds to an RCP generated from the biological sample in a plurality of detection cycles. In some embodiments, the Argonaute-barcode-binding probe complexes that are not bound are removed from the sample (for example, performing one or more wash steps). In some embodiments, the method further comprises contacting the biological sample with different populations of Argonaute-barcode-binding probe complexes in sequential cycles. In some embodiments, in each cycle, a signal associated with the Argonaute-barcode-binding probe complexes are recorded at a location in the biological sample, thereby generating a signal code (i.e., a series of signals) corresponding to the plurality of barcode subunits on a probe. In some embodiments, the signal code comprises the signal(s) (e.g., level of signals) recorded at the location in each of the sequential cycles. In some embodiments, the signal code comprises distinguishable signals recorded at the location in each of the sequential cycles. In some embodiments, the method further comprises using the signal code to identify the target analyte bound by the barcode probe of the barcode probe library at the location in the biological sample.

In some embodiments, the bound Argonaute-barcode-binding probe complexes, and/or the signals associated with the Argonaute-barcode-binding probe complexes, are removed from the biological sample in between sequential detection cycles. In some embodiments, removing the bound Argonaute-barcode-binding probe complexes comprises chemically stripping the Argonaute-barcode-binding probe complexes from the biological sample. In some embodiments, removing the bound Argonaute-barcode-binding probe complexes comprises contacting the biological sample with competitor oligonucleotides that compete with and displace the barcode-binding probes. In some embodiments, removing the signal associated with the Argonaute-barcode-binding probe complexes comprises quenching fluorophores associated with the Argonaute-barcode binding probe complexes. In some embodiments, removing the signal associated with the Argonaute-barcode-binding probe complexes comprises cleaving fluorophores from the Argonaute-barcode binding probe complexes. In some embodiments, cleaving the fluorophores comprises enzymatically cleaving the fluorophores. In some embodiments, cleaving the fluorophores comprises chemically cleaving the fluorophores.

In some embodiments, the biological sample is contacted with at least two subsets of Argonaute-barcode-binding probe complexes of a pool of Argonaute-barcode-binding probe complexes, wherein each subset comprises at least one different barcode-binding probes. For example, a first subset of barcode-binding probes is provided in a first detection cycle and subsequently a second subset of barcode-binding probes is provided in a second detection cycle, wherein the first subset of barcode-binding probes comprises at least one barcode-binding probe that does not have the same barcode-binding domain as a barcode-binding probe of the second subset of barcode-binding probes. In some cases, a first subset of barcode-binding probes is provided in a first detection cycle and subsequently a second subset of barcode-binding probes is provided in a second detection cycle, wherein the first subset of barcode-binding probes comprises at least one barcode-binding probe that is the same as a barcode-binding probe of the second subset of barcode-binding probes. In some embodiments, a plurality of barcode-binding probes are used for detection of barcode subunits in the barcode probe library. In some embodiments, the barcode-binding domain is in the 3′ tail region of the barcode-binding probe. In some embodiments, the barcode-binding domain is in the 5′ tail region of the barcode-binding probe. In some embodiments, in each of the sequential cycles, different Argonaute-barcode-binding probe complexes are hybridized to the same barcode subunit sequence. In some embodiments, in two or more of the sequential cycles, different Argonaute-barcode-binding probe complexes are hybridized to different barcode subunit. In some embodiments, the plurality of barcode-binding probes further comprises at least one additional barcode-binding probe that is not in a complex with an Argonaute protein.

In some embodiments, the method comprises contacting the biological sample with an Argonaute-barcode-binding probe complex that binds to the RCP. In some embodiments, the method further comprises removing molecules of Argonaute-barcode-binding probe complexes that are not bound to the RCP. In some embodiments, the method further comprises contacting the biological sample with detectably labeled probes that bind the Argonaute-barcode-binding probe complex. In some embodiments, in each cycle, a detectably labeled Argonaute-barcode-binding probe complex is bound to a barcode subunit sequence in the RCP and a signal (e.g., a level of signal) associated with the Argonaute-barcode-binding probe complex is recorded at a location in the biological sample, thereby generating a signal code sequence corresponding to the barcode subunit. In some embodiments, in two or more of the sequential cycles, different Argonaute-barcode-binding probe complexes bind to a barcode subunit sequence. In some embodiments, in each of the sequential cycles for decoding the barcode subunits, the Argonaute-barcode-binding probe complex binds to the sequence, is observed, and then is subsequently removed from the barcode subunit. In some embodiments, the removal of the Argonaute barcode-binding probe complex is performed via chemical stripping (e.g., formamide stripping). In some embodiments, the removal of the Argonaute-barcode-binding complex is performed via a toehold strand displacement approach, in which competitor oligonucleotides are designed and used to displace the guide RNAs that are in the Argonaute-barcode-binding probe complex from the sequence. In some embodiments, each pair of adjacent barcode subunits in the probe or a generated amplification product thereof comprising complements of the barcode subunits are partially overlapping. In some embodiments, each pair of adjacent barcode subunits in the barcode probe or a generated amplification product thereof comprising complements of the barcode subunits overlap by between 2 and 5 nucleotides.

In any of the embodiments herein, the detecting step comprises contacting the biological sample with one or more Argonaute-barcode-binding probe complexes that binds to the barcode subunit sequences or complements thereof (e.g., of the plurality of probes or probe sets or rolling circle amplification product generated using the plurality of probes or probe sets), wherein the Argonaute-barcode-binding probe complexes are detectably labeled. In any of the embodiments herein, the detecting step can further comprise removing the one or more Argonaute-barcode-binding probe complexes from the barcode subunit sequences or complements thereof (e.g., of the plurality of probes or probe sets or rolling circle amplification product generated using the plurality of probes or probe sets). In any of the embodiments herein, the contacting and removing steps can be repeated. In some cases, the repeated contacting, detection and removing steps allows detection of barcode subunit sequences or complements thereof and identification of the corresponding sequences of signal codes (e.g., fluorophore sequences that identifies the target analyte). In some embodiments, after detection, the signal associated with the barcode subunit is removed (e.g., by quenching, cleaving the label, and/or performing one or more washes).

In some embodiments, the signal code sequence comprises the signals (e.g., level of signals) at the location in each of the sequential cycles. In some embodiments, the method provided herein further comprises using the signal code sequence to identify the target analyte bound by the probe at the location in the biological sample.

In some embodiments, a signal associated with a barcode-binding probe in a complex with a nuclease-deficient Argonaute protein is measured and quantitated. The terms “label” and “detectable label” comprise a directly or indirectly detectable moiety that is associated with (e.g., conjugated to) a molecule to be detected, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, Y Pet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, techniques and methods methodologies applicable to the provided embodiments comprise those described in, for example, U.S. Pat Nos. 4,757,141, 5,151,507, and 5,091,519, the content of each of which are herein incorporated by reference for all purposes. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), the content of each of which are herein incorporated by reference for all purposes. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, U.S. Pat. Pub. No. 2002/0045045, and U.S. Pat. Pub. No. 2003/0017264, the content of each of which are herein incorporated by reference for all purposes. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, the barcode-binding probe in a complex with a nuclease-deficient Argonaute protein disclosed herein comprises one or more detectably labeled, e.g., fluorescent, nucleotides. Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (A mersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-I2-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). For examples of methods for custom synthesis of nucleotides having other fluorophores, see, Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (see Lakowicz et al. (2003) Bio Techniques 34:62, which is hereby incorporated by reference in its entirety).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160, the content of each of which are herein incorporated by reference for all purposes. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting comprises performing microscopy, scanning mass spectrometry or other imaging techniques described herein. In such aspects, the detecting comprises determining a signal, e.g., a fluorescent signal. In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the barcode-binding probe in a complex with a nuclease-deficient Argonaute protein disclosed herein. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the barcode-binding probe in a complex with a nuclease-deficient Argonaute protein disclosed herein. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FM M), feature-oriented scanning probe microscopy (FOSPM), kelvin probe force microscopy (K PFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SV M), and synchrotron x-ray scanning tunneling microscopy (SXSTM), and intact tissue expansion microscopy (exM).

### III. Samples, Analytes, and Target Sequences

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism comprises one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprises a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein can be any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

In some cases, a biological sample is harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed.

In some embodiments, multiple sections are obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. In some embodiments, a frozen tissue sample is sectioned, e.g., thinly sliced, onto a substrate surface using any number of suitable methods. For example, a tissue sample is prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization. In some embodiments, processing of the biological sample, such as de-waxing, allows the biological sample to become permeabilized.

As an alternative to formalin fixation described above, in some embodiments, a biological sample is fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). M ore generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural - - - attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample is stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain is specific to proteins, phospholipids, DNA (e.g., dsDNA, SSDNA), RNA, an organelle or compartment of the cell. In some embodiments, the sample is contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H & E).

The sample can be stained using hematoxylin and eosin (H & E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, a biological sample is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### B. Analytes

A biological sample may comprise one or a plurality of analytes of interest. Methods for performing multiplexed assays to analyze two or more different analytes in a single biological sample are provided. The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, SiRNA, snoRNAs, piRNA, tRNA-derived small RNA (tsRNA), and small rDNA-derived RNA (srRNA). The RNA can be double-stranded RNA or single-stranded RNA. In some embodiments, the RNA comprises circular RNA. In some embodiments, the RNA is a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In some embodiments, the plurality of target analytes comprise a plurality of cellular RNA analytes or a product thereof. In some embodiments, a product thereof is a cDNA product of the cellular RNA analyte. In some embodiments, the cDNA products of the cellular RNA analytes are generated by reverse transcription in the biological sample before contacting the sample with the barcode probe library.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. In some embodiments, the label is conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### IV. Compositions, Kits, and Systems

Provided herein are kits, for example comprising one or more oligonucleotides and proteins, e.g., any described in Sections I-III, and instructions for performing the methods provided herein. In some embodiments, the kits further comprise one or more reagents for performing the methods provided herein. In some embodiments, the kits further comprise one or more reagents required for one or more steps comprising hybridization, ligation, extension, amplification, detection, and/or sample preparation as described herein. In some embodiments, the kit comprises any one or more of the barcode probe or barcode probe sets (e.g., padlock probes) of the barcode probe library, the Argonaute-barcode-binding probe complexes, and/or detectably labeled oligonucleotides disclosed herein. In some embodiments, any or all of the oligonucleotides are DNA molecules. In some embodiments, the kit further comprises an enzyme such as a ligase and/or a polymerase described herein. In some embodiments, the kit comprises a polymerase, for instance for performing extension of the primers to generate an amplification product. In some embodiments, the kits may contain reagents for forming a functionalized matrix (e.g., a hydrogel), such as any suitable functional moieties. The various components of the kit may be present in separate containers or certain compatible components may be pre-combined into a single container. In some embodiments, the kits further contain instructions for using the components of the kit to practice the provided methods.

In some embodiments, provided herein is a kit for analyzing a biological sample, comprising a barcode probe library to provide a plurality of probes bound to target analytes, wherein each barcode probe of the barcode probe library comprises (i) a plurality of barcode subunits and (ii) a region that binds to a target analyte; wherein each barcode subunit is 10-30 nucleotides in length and the plurality of barcode subunits of the barcode probe library has a total of at least 50 different barcode subunits; and a plurality of barcode-binding probes, wherein each barcode-binding probe is in a complex with a nuclease-deficient Argonaute protein and each barcode-binding probes comprises a barcode-binding domain that binds to a sequence of the barcode subunit of the plurality of barcode subunits of a probe or a complement thereof. In some embodiments, the kit comprises one or more reagents for generating a rolling circle amplification product (RCP) of the barcode probe (e.g., circular probe or circularizable probe or probe set), wherein the RCP comprises multiple copies of the plurality of barcode subunits. In some embodiments, the kit comprises a mixture of preloaded Argonaute-barcode-binding probe complexes.

In some embodiments, a kit disclosed herein comprises a pool of Argonaute-barcode-binding probe complexes each comprising a detectable label. In some embodiments, the biological sample is imaged to detect signals associated with the detectable labels at locations in the biological sample, thereby detecting one or more of the plurality of barcode subunits in the probes bound to biological sample or complements thereof. In some embodiments, the one or more of the plurality of different barcode subunits are identified in the biological sample, based on the signals detected at the locations.

In some aspects, a kit disclosed herein comprises an Argonaute and barcode-binding probe complex comprising a detectably labeled Argonaute protein (e.g., a fluorescently labeled Argonaute as shown in the right panel of FIG. 1B). In some aspects, a kit disclosed herein comprises an Argonaute-barcode-binding probe complex comprising a directly labeled barcode-binding probe (e.g., as shown in the left panel of FIG. 1B). In some aspects, a kit disclosed herein comprises an Argonaute-barcode-binding probe complex comprising an indirectly labeled barcode-binding probe (e.g., as shown in the middle panel of FIG. 1B), wherein the Argonaute-barcode-binding probe complex is bound to a separate detectably labeled probe. In some embodiments, the Argonaute-barcode-binding probe complex comprises a detectable moiety (e.g., a fluorescent label) that allows for it to be detected in situ at a location in the biological sample when bound to an RCP generated from the biological sample. In some embodiments, the barcode-binding probe of the Argonaute-barcode-binding probe complex comprises a detectable label, optionally wherein the detectable label is attached to the 3′ tail region. In some embodiments, the kit comprises a plurality of pools of Argonaute-barcode-binding probe complexes. In some embodiments, the kit comprises a plurality of different mixtures of Argonaute-barcode-binding probe complexes.

In some embodiments, the kits comprises reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit also comprises any of the reagents described herein, e.g., wash buffer and ligation buffer. In some embodiments, the kits contain reagents for detection and/or sequencing, such as detectably labeled oligonucleotides or detectable labels. In some embodiments, the kits optionally contain other components, for example nucleic acid primers, enzymes and reagents, buffers, nucleotides, modified nucleotides, and/or reagents for additional assays.

In some embodiments, provided herein are systems, for example comprising a biological sample, and one or more oligonucleotides and proteins, (e.g., any described in Sections I-III). In some embodiments, the systems further comprise one or more reagents for performing the methods provided herein. In some embodiments, the systems further comprise one or more reagents required for one or more steps comprising hybridization, ligation, extension, amplification, detection, and/or sample preparation as described herein. In some embodiments, the system comprises any one or more of the barcode probe or barcode probe sets (e.g., padlock probes) of the barcode probe library, the Argonaute-barcode-binding probe complexes, and/or detectably labeled oligonucleotides disclosed herein. In some embodiments, any or all of the oligonucleotides are DNA molecules. In some embodiments, the system further comprises an enzyme such as a ligase and/or a polymerase described herein. In some embodiments, the system comprises a polymerase, for instance for performing extension of the primers to generate an amplification product. In some embodiments, the systems may contain reagents for forming a functionalized matrix (e.g., a hydrogel), such as any suitable functional moieties.

In some embodiments, provided herein is a system for analyzing a biological sample, comprising the biological sample; a barcode probe library comprising a plurality of probes, wherein each barcode probe of the barcode probe library comprises i) a plurality of barcode subunits and ii) a region that binds to a target analyte; wherein each barcode subunit is 10-30 nucleotides in length and the plurality of barcode subunits of the barcode probe library has a total of at least 50 different barcode subunits; and a plurality of barcode-binding probes, wherein each barcode-binding probe is in a complex with a nuclease-deficient Argonaute protein and each barcode-binding probes comprises a barcode-binding domain that binds to a sequence of the barcode subunit of the plurality of barcode subunits of a probe or a complement thereof. In some embodiments, the system comprises one or more reagents for generating a rolling circle amplification product (RCP) of the barcode probe (e.g., circular probe or circularizable probe or probe set), wherein the RCP comprises multiple copies of the plurality of barcode subunits. In some embodiments, the system comprises a mixture of preloaded Argonaute-barcode-binding probe complexes.

In some embodiments, a system disclosed herein comprises a pool of Argonaute-barcode-binding probe complexes each comprising a detectable label, and a biological sample. In some embodiments, the biological sample is imaged to detect signals associated with the detectable labels at locations in the biological sample, thereby detecting one or more of the plurality of barcode subunits in the probes bound to biological sample or complements thereof. In some embodiments, the one or more of the plurality of different barcode subunits are identified in the biological sample, based on the signals detected at the locations.

In some aspects, a system disclosed herein comprises a biological sample and an Argonaute and barcode-binding probe complex comprising a detectably labeled Argonaute protein (e.g., a fluorescently labeled Argonaute as shown in the right panel of FIG. 1B). In some aspects, a system disclosed herein comprises a biological sample and an Argonaute-barcode-binding probe complex comprising a directly labeled barcode-binding probe (e.g., as shown in the left panel of FIG. 1B). In some aspects, a system disclosed herein comprises a biological sample and an Argonaute-barcode-binding probe complex comprising an indirectly labeled barcode-binding probe (e.g., as shown in the middle panel of FIG. 1B), wherein the Argonaute-barcode-binding probe complex is bound to a separate detectably labeled probe. In some embodiments, the Argonaute-barcode-binding probe complex comprises a detectable moiety (e.g., a fluorescent label) that allows for it to be detected in situ at a location in the biological sample when bound to an RCP generated from the biological sample. In some embodiments, the barcode-binding probe of the Argonaute-barcode-binding probe complex comprises a detectable label, optionally wherein the detectable label is attached to the 3′ tail region. In some embodiments, the system comprises a plurality of pools of Argonaute-barcode-binding probe complexes. In some embodiments, the system comprises a plurality of different mixtures of Argonaute-barcode-binding probe complexes.

In some embodiments, the system comprises reagents and/or consumables required for performing one or more steps of the provided methods. In some embodiments, the systems comprise reagents for fixing, embedding, and/or permeabilizing the biological sample. In some embodiments, the systems comprise reagents, such as enzymes and buffers for ligation and/or amplification, such as ligases and/or polymerases. In some aspects, the system also comprises any of the reagents described herein, e.g., wash buffer and ligation buffer. In some embodiments, the systems contain reagents for detection and/or sequencing, such as detectably labeled oligonucleotides or detectable labels. In some embodiments, the systems optionally contain other components, for example nucleic acid primers, enzymes and reagents, buffers, nucleotides, modified nucleotides, and/or reagents for additional assays.

### V. Opto-Fluidic Instruments for Analysis of Biological Samples

Provided herein is an instrument having integrated optics and fluidics modules (an “opto-fluidic instrument” or “opto-fluidic system”) for detecting target analytes in biological samples (e.g., one or more cells or a tissue sample) as described herein. In an opto-fluidic instrument, the fluidics module is configured to deliver one or more reagents (e.g., barcode-binding probe in a complex with a nuclease-deficient Argonaute protein) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., detected as described in Section II). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target analyte detection via probe hybridization (e.g., barcode-binding probe in a complex with a nuclease-deficient Argonaute protein), the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

In various embodiments, the opto-fluidic instrument may be in communication with a cloud computing platform. In various embodiments, the opto-fluidic instrument analyzes the sample and generates an output that includes indications of the presence of the target molecules in the sample. For instance, the opto-fluidic instrument employs a hybridization technique for detecting molecules (e.g., using barcode-binding probes is in a complex with a nuclease-deficient Argonaute protein), wherein the opto-fluidic instrument performs successive detection cycles of providing and binding the subsets of barcode-binding probes (e.g., using two or more subsets of barcode-binding probes, where a barcode-binding probe in a complex with a nuclease-deficient Argonaute protein is excited by a color channel) and imaging the sample to detect target analytes in the probed sample. In some embodiments, the output includes optical signatures (e.g., a codeword) specific to each target analyte, which allow the identification of the target analytes probed by the barcode probe library.

### VI. Applications

In some aspects, the provided embodiments are applied in an in situ method of analyzing nucleic acid sequences in intact tissues or samples in which the spatial information has been preserved. In some aspects, the embodiments are applied in an imaging or detection method for multiplexed nucleic acid analysis. In some aspects, the target nucleic acid is an RNA. In some embodiments, the target nucleic acid is an mRNA. In some aspects, the target nucleic acid is a DNA. In some embodiments, the target nucleic acid is genomic DNA. In some aspects, the target nucleic acid is cDNA.

In some aspects, the embodiments are applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples. In some aspects, the embodiments can be applied to visualize the distribution of genetically encoded markers in whole tissue at subcellular resolution.

### VII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Detecting Barcode Subunits In Situ

This example describes a workflow wherein a plurality of circularizable barcode probes (e.g., padlock probes) hybridize to corresponding target nucleic acid analytes and is used to generate rolling circle amplification products (RCPs) comprising copies of the barcode subunit sequences of each probe. The barcode subunits are detected by contacting the RCPs with a plurality of barcode-binding probes in complexes with nuclease-deficient Argonaute proteins.

A tissue sample comprising a target nucleic acid (e.g., mRNA) is sectioned and the tissue sections are mounted on a slide, fixed (e.g., by incubating in paraformaldehyde (PFA)), washed, and permeabilized (e.g., using Triton-X). After permeabilization, the tissue sections are washed, dehydrated, and rehydrated.

The tissue sample is contacted with a barcode probe library targeting at least 200 target analytes. A circularizable barcode probe or barcode probe set comprises a region that binds to a target analyte and a plurality of barcode subunits, as shown in FIG. 1A. Each barcode subunit of the barcode probe or barcode probe set is 15 nucleotides in length and each barcode probe comprises at least three barcode subunits. To select the sequences for the barcode subunits, potential interaction(s) between sequences of the amplification products (e.g., RCPs) of the barcode probes, a plurality of barcode-binding probes and a plurality of detectably labeled probes (e.g., that binds to the 3′ tail sequence of the barcode-binding probe) are analyzed. In some cases, each barcode subunit comprises a common domain at the 5′ and 3′ end of the barcode subunit. The circularizable barcode probe or probe set is ligated to form a circularized barcode probe that is used for as a template for rolling circle amplification (RCA). For RCA, the tissue sections are washed and then incubated in an RCA reaction mixture (containing Phi29 reaction buffer, dNTPs, Phi29 polymerase) to generate RCPs containing the multiple copies of the plurality of barcode subunits.

As shown in FIG. 1A, provided barcode-binding probes are in complex with Argonaute-and bind to the RCPs in each detection cycle. The barcode-binding probes comprise a 5′ seed region for binding to barcode subunit sequences. The tissue sections containing RCPs are incubated with the Argonaute-barcode-binding probe complexes in a buffer comprising Mg²⁺ to allow binding of Argonaute-barcode-binding probe complexes to the RCPs. As shown in the middle panel of FIG. 1B, Argonaute-barcode-binding probe complexes are indirectly labeled using a fluorescently labeled probe that binds to an overhang of the barcode-binding probe. A first set of Argonaute-barcode-binding probes comprise barcode-binding probes with a seed region for binding a first barcode subunit and a first detectable label (e.g., a green fluorescent moiety such as eGFP). In a second detection cycle, a second set barcode-binding probes in complex with Argonaute proteins are associated with a second detectable label (e.g., a red fluorescent moiety such as mCherry) are subsequently detected in situ. The tissue sections are washed in between detection cycles. Each signal associated with a bound barcode-binding probes detected in each cycle is used to obtain a signal of the signal code that collectively identifies the target analyte.

In some aspects, to detect 200 target analytes as described, the barcode probe library described herein uses selected barcode subunit sequences and barcode-binding probe sequences in complexes with Argonaute proteins that improve binding and thus have a lower tolerance for non-matching interactions (e.g., off-target binding) compared to sequences that are used as free oligonucleotides where binding occurs without the aid of the Argonaute protein.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for detecting a target analyte in a biological sample, comprising:
(a) contacting the biological sample comprising a plurality of target analytes with a barcode probe library comprising a plurality of barcode probes, wherein each barcode probe of the barcode probe library comprises (i) a plurality of barcode subunits and (ii) a region that binds to a target analyte of the plurality of target analytes,
wherein each barcode subunit is 10-30 nucleotides and the plurality of barcode subunits of the barcode probe library has a total of at least 50 different barcode subunits,
wherein the plurality of barcode subunits on a barcode probe of the barcode probe library identifies the target analyte, and
wherein each target analyte is assigned a signal code that identifies the target analyte; and
(b) detecting the plurality of barcode subunits in the barcode probes bound to target analytes in a plurality of detection cycles using a plurality of barcode-binding probes to obtain the signal code, wherein each detection cycle comprises:
contacting the biological sample with at least a subset of the plurality of barcode-binding probes, wherein each barcode-binding probe of at least the subset of the plurality of barcode-binding probes is in a complex with a nuclease-deficient Argonaute protein and each barcode-binding probe comprises a barcode-binding domain that binds to a sequence of the barcode subunit of the plurality of barcode subunits of a barcode probe or a complement thereof; and
detecting a signal associated with a bound barcode-binding probe to obtain a signal of the signal code;
thereby determining the identity of the target analyte using the detected signal code.

2. The method of claim 1, wherein the barcode probe library has a total of at least 60 different barcode subunits.

3-6. (canceled)

7. The method of claim 1, wherein the nuclease-deficient Argonaute protein is a DNA-guided Argonaute, and the barcode probes of the barcode probe library comprise DNA.

8-12. (canceled)

13. The method of claim 1, wherein the barcode-binding probe and the nuclease-deficient Argonaute protein are bound in the complex before contacting the biological sample.

14. The method of claim 1, wherein the barcode-binding probe and the nuclease-deficient Argonaute protein form a complex in the biological sample.

15. The method of claim 1, wherein the barcode subunits of the plurality of barcode subunits comprise artificial sequences with less than 70% homology to an endogenous human or mouse sequence.

16-20. (canceled)

21. The method of claim 1, wherein the nuclease-deficient Argonaute protein is labeled with a detectable moiety.

22. The method of claim 1, wherein the barcode-binding probes are labeled with a detectable moiety.

23. The method of claim 1, wherein the barcode-binding probes are not directly labeled with a fluorescent dye.

24-25. (canceled)

26. The method of claim 1, wherein the plurality of barcode subunits of each barcode probe of the barcode probe library are overlapping.

27. (canceled)

28. The method of claim 1, wherein each barcode subunit is 10-20 nucleotides in length.

29-30. (canceled)

31. The method of claim 1, comprising washing the biological sample between contacting the biological sample with different subsets of barcode-binding probes from the plurality of barcode-binding probes.

32. (canceled)

33. The method of claim 1, wherein the method comprises generating a plurality of amplification products of the plurality of probes bound to the target analytes before detecting the plurality of barcode subunits.

34. The method of claim 1, wherein the method comprises circularizing the plurality of barcode probes bound to target analytes prior to generating the plurality of amplification products.

35-39. (canceled)

40. The method of claim 1, wherein the barcode-binding domain is between about 14 and 20 nucleotides in length.

41-42. (canceled)

43. The method of claim 1, wherein detecting the plurality of barcode subunits comprises imaging the biological sample.

44-48. (canceled)

49. The method of claim 1, wherein the detecting is performed on a cell or tissue sample.

50. The method of claim 1, wherein the target analytes of the plurality of target analytes comprise a plurality of cellular RNA analytes or a product thereof.

51. The method of claim 1, wherein the target analytes of the plurality of target analytes are associated with a non-nucleic acid analyte.

52-60. (canceled)

61. A kit, comprising:
a barcode probe library comprising a plurality of barcode probes, wherein each barcode probe of the barcode probe library comprises (i) a plurality of barcode subunits and (ii) a region that binds to a target analyte;
wherein each barcode subunit is 10-30 nucleotides in length and the plurality of barcode subunits of the barcode probe library has a total of at least 50 different barcode subunits; and
a plurality of barcode-binding probes,
wherein each barcode-binding probe is in a complex with a nuclease-deficient Argonaute protein and each barcode-binding probe comprises a barcode-binding domain that binds to a sequence of the barcode subunit of the plurality of barcode subunits of a barcode probe or a complement thereof.

62-92. (canceled)
